SEQUENCE LISTING (85 sequences)

SEQ ID NO: 1 - DNA, 355 bp, Homo sapiens
  Clone PR087-29B5 IL38 VH nucleic acid
gtgaaggtctcctgcaaggcttctggttacagctttatcaactatggcatcacctgggtgcgccaggtccctggacaagggcttgagtggatgggatggatcagcgcttacaatgttaagacaaagtatgcaccgaagttccagggcagagtcaacatgaatacagacacatccacgaggacagcctacatggagctgaggagcctgagatctgacgacacggccgtctattactgtgcgagagatagggattacgatttttggagtggttcggcttttgatatctggggccaagggacaatggtcaccgtctcttcagcctccaccaagggcccatcggtcttccccctggcgc

SEQ ID NO: 2 - protein, 118 aa, Homo sapiens
  Clone PR087-29B5 IL38 VH amino acid
VKVSCKASGYSFINYGITWVRQVPGQGLEWMGWISAYNVKTKYAPKFQGRVNMNTDTSTRTAYMELRSLRSDDTAVYYCARDRDYDFWSGSAFDIWGQGTMVTVSSASTKGPSVFPLA

SEQ ID NO: 3 - DNA, 308 bp, Homo sapiens
  Clone PR087-29B5 IL38 VL nucleic acid
aagagtcaccctctcctgcagggccagtcagagtattagcaccaactacttagcctggtaccagcagaaacctggccaggctcccagtctactcatctatggtgcatccagcagggccactggcatcccagacaggttcagtggcagtgggtctgggacagacttcactctcaccatcagcagactggagcctgaagattttgcagtgtattactgtcagcagtatggtagctcacctccgaggacttttggccaggggaccaagctggagatcagacgaactgtggctgcaccatctgtcttcatct

SEQ ID NO: 4 - protein, 102 aa, Homo sapiens
  Clone PR087-29B5 IL38 VL amino acid
RVTLSCRASQSISTNYLAWYQQKPGQAPSLLIYGASSRATGIPDRFSGSGSGTDFTLTISRLEPEDFAVYYCQQYGSSPPRTFGQGTKLEIRRTVAAPSVFI

SEQ ID NO: 5 - DNA, 369 bp, Homo sapiens
  IMM20130 IL38 VH nucleic acid
caggttcagctggttcagtctggcgccgaagtgaagaaacctggcgcctctgtgaaggtgtcctgcaaggccagcggctacagcttcatcaactacggcatcacctgggtccgacaggtgccaggacaaggcttggaatggatgggctggatcagcgcctacaacgtcaagaccaaatacgcccctaagttccagggccgcgtgaacatgaacaccgacaccagcaccagaaccgcctacatggaactgcggagcctgagatccgatgacaccgccgtgtactactgcgccagagacagagactacgacttttggagcggcagcgccttcgatatctggggccagggaacaatggtcaccgtgtctagt

SEQ ID NO: 6 - DNA, 369 bp, Homo sapiens; identical to SEQ ID NO: 5
  IMM20130 IL38 VH codon optimized expression fragment nucleic acid

SEQ ID NO: 7 - protein, 123 aa, Homo sapiens
  IMM20130 IL38 VH amino acid
QVQLVQSGAEVKKPGASVKVSCKASGYSFINYGITWVRQVPGQGLEWMGWISAYNVKTKYAPKFQGRVNMNTDTSTRTAYMELRSLRSDDTAVYYCARDRDYDFWSGSAFDIWGQGTMVTVSS

SEQ ID NO: 8 - DNA, 324 bp, Homo sapiens
  IMM20130 IL38 VL nucleic acid
gagatcgtgctgacacagagccctggcacactgtcactgtctccaggcgagagagtgaccctgagctgtagagccagccagagcatcagcaccaactacctggcctggtatcagcagaagcctggacaggctcctagcctgctgatctacggcgcctcttctagagccacaggcatccccgatagattcagcggctctggcagcggcaccgatttcaccctgacaatcagcagactggaacccgaggacttcgccgtgtactactgtcagcagtacggcagcagccctcctagaacatttggccagggcaccaagctggaaatc

SEQ ID NO: 9 - DNA, 327 bp, Homo sapiens
  IMM20130 IL38 VL codon optimized expression fragment nucleic acid
gagatcgtgctgacacagagccctggcacactgtcactgtctccaggcgagagagtgaccctgagctgtagagccagccagagcatcagcaccaactacctggcctggtatcagcagaagcctggacaggctcctagcctgctgatctacggcgcctcttctagagccacaggcatccccgatagattcagcggctctggcagcggcaccgatttcaccctgacaatcagcagactggaacccgaggacttcgccgtgtactactgtcagcagtacggcagcagccctcctagaacatttggccagggcaccaagctggaaatcaag

SEQ ID NO: 10 - protein, 109 aa, Homo sapiens
  IMM20130 IL38 VL amino acid
EIVLTQSPGTLSLSPGERVTLSCRASQSISTNYLAWYQQKPGQAPSLLIYGASSRATGIPDRFSGSGSGTDFTLTISRLEPEDFAVYYCQQYGSSPPRTFGQGTKLEIK

SEQ ID NO: 11 - DNA, 1365 bp, Homo sapiens
  IMM20130 IL38 HC nucleic acid
caggttcagctggttcagtctggcgccgaagtgaagaaacctggcgcctctgtgaaggtgtcctgcaaggccagcggctacagcttcatcaactacggcatcacctgggtccgacaggtgccaggacaaggcttggaatggatgggctggatcagcgcctacaacgtcaagaccaaatacgcccctaagttccagggccgcgtgaacatgaacaccgacaccagcaccagaaccgcctacatggaactgcggagcctgagatccgatgacaccgccgtgtactactgcgccagagacagagactacgacttttggagcggcagcgccttcgatatctggggccagggaacaatggtcaccgtgtctagtgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 12 - protein, 453 aa, Homo sapiens
  IMM20130 IL38 HC amino acid
QVQLVQSGAEVKKPGASVKVSCKASGYSFINYGITWVRQVPGQGLEWMGWISAYNVKTKYAPKFQGRVNMNTDTSTRTAYMELRSLRSDDTAVYYCARDRDYDFWSGSAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 654 bp, Homo sapiens
  IMM20130 IL38 LC nucleic acid
gagatcgtgctgacacagagccctggcacactgtcactgtctccaggcgagagagtgaccctgagctgtagagccagccagagcatcagcaccaactacctggcctggtatcagcagaagcctggacaggctcctagcctgctgatctacggcgcctcttctagagccacaggcatccccgatagattcagcggctctggcagcggcaccgatttcaccctgacaatcagcagactggaacccgaggacttcgccgtgtactactgtcagcagtacggcagcagccctcctagaacatttggccagggcaccaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 14 - protein, 216 aa, Homo sapiens
  IMM20130 IL38 LC amino acid
EIVLTQSPGTLSLSPGERVTLSCRASQSISTNYLAWYQQKPGQAPSLLIYGASSRATGIPDRFSGSGSGTDFTLTISRLEPEDFAVYYCQQYGSSPPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 13 aa, Homo sapiens
  IMM20130 IL38 VH-CDR1 amino acid
KASGYSFINYGIT

SEQ ID NO: 16 - protein, 10 aa, Homo sapiens
  IMM20130 IL38 VH-CDR2 amino acid
WISAYNVKTK

SEQ ID NO: 17 - protein, 16 aa, Homo sapiens
  IMM20130 IL38 VH-CDR3 amino acid
ARDRDYDFWSGSAFDI

SEQ ID NO: 18 - protein, 12 aa, Homo sapiens
  IMM20130 IL38 VL-CDR1 amino acid
RASQSISTNYLA

SEQ ID NO: 19 - protein, 8 aa, Homo sapiens
  IMM20130 IL38 VL-CDR2 amino acid
YGASSRAT

SEQ ID NO: 20 - protein, 10 aa, Homo sapiens
  IMM20130 IL38 VL-CDR3 amino acid
QQYGSSPPRT

SEQ ID NO: 21 - DNA, 352 bp, Mus sp.
  Clone M3 IL38-C VH nucleic acid
gaggtccagctgcaacagtctggacctgagctggtgaagcctggggcttcagtgaaaataccctgcaaggcttctggatacacattcactgactacaatatggactgggtgaagcagagccatggaaagagccttgagtggattggagatattaatcctaacaatggtggtactatctacaaccagaagttcaagggcaaggccacattgactgtagacaagtcttccagcacagcctacatggagctccgcagcctgacatctgaggacactgcagtctattactgttcaagaccctattatggttactttgcttactggggccaagggactctggtcactgtctctgcag

SEQ ID NO: 22 - protein, 117 aa, Mus sp.
  Clone M3 IL38-C VH amino acid
EVQLQQSGPELVKPGASVKIPCKASGYTFTDYNMDWVKQSHGKSLEWIGDINPNNGGTIYNQKFKGKATLTVDKSSSTAYMELRSLTSEDTAVYYCSRPYYGYFAYWGQGTLVTVSA

SEQ ID NO: 23 - protein, 13 aa, Mus sp.
  Clone M3 IL38-C VH CDR1 amino acid
KASGYTFTDYNMD

SEQ ID NO: 24 - protein, 10 aa, Mus sp.
  Clone M3 IL38-C VH CDR2 amino acid
DINPNNGGTI

SEQ ID NO: 25 - protein, 10 aa, Mus sp.
  Clone M3 IL38-C VH CDR3 amino acid
SRPYYGYFAY

SEQ ID NO: 26 - DNA, 337 bp, Mus sp.
  Clone M3 IL38-C VH Alternate nucleic acid
gaggtccagctgcaacagtctggacctgagttggtgaagcctggggcttcagtgaagatgtcctgcaaggcttctggctacacattcactgactactacatacactgggtgaagcagagccatggaaagggccttgagtggattggatatatttttcctaataatggtggtaatggctacagccagaagttcaagggcaaggccacaatgactgtagacaagtcctccagcacagcctacatggagctccgcagcctgacatctgaggactctgcagtctattattgtgcaagatttgtttactggggccaagggacgctggtcactgtctctgcag

SEQ ID NO: 27 - protein, 112 aa, Mus sp.
  Clone M3 IL38-C VH Alternate amino acid
EVQLQQSGPELVKPGASVKMSCKASGYTFTDYYIHWVKQSHGKGLEWIGYIFPNNGGNGYSQKFKGKATMTVDKSSSTAYMELRSLTSEDSAVYYCARFVYWGQGTLVTVSA

SEQ ID NO: 28 - protein, 13 aa, Mus sp.
  Clone M3 IL38-C VH Alternate CDR1 amino acid
KASGYTFTDYYIH

SEQ ID NO: 29 - protein, 10 aa, Mus sp.
  Clone M3 IL38-C VH Alternate CDR2 amino acid
YIFPNNGGNG

SEQ ID NO: 30 - protein, 11 aa, Mus sp.
  Clone M3 IL38-C VH Alternate CDR3 amino acid
ARGGYDAGFVY

SEQ ID NO: 31 - DNA, 355 bp, Mus sp.
  Clone M8 IL38-C VH nucleic acid
gaggtccarctgcaacagtctggacctgagttggtgaagcctggggcttcagtgaagatgtcctgcaaggcttctggctacacattcactgactactacatacactgggtgaagcagagccatggaaagggccttgagtggattggatatatttttcctaataatggtggtaatggctacagccagaagttcaagggcaaggccacaatgactgtagacaagtcctccagcacagcctacatggagctccgcagcctgacatctgaggactctgcagtctattattgtgcaagagggggctacgacgcggggtttgtttactggggccaagggacgctggtcactgtctctgcag

SEQ ID NO: 32 - protein, 118 aa, Mus sp.
  Clone M8 IL38-C VH amino acid
EVQLQQSGPELVKPGASVKMSCKASGYTFTDYYIHWVKQSHGKGLEWIGYIFPNNGGNGYSQKFKGKATMTVDKSSSTAYMELRSLTSEDSAVYYCARGGYDAGFVYWGQGTLVTVSA

SEQ ID NO: 33 - protein, 13 aa, Mus sp.
  Clone M8 IL38-C VH CDR1 amino acid
KASGYTFTDYYIH

SEQ ID NO: 34 - protein, 10 aa, Mus sp.
  Clone M8 IL38-C VH CDR2 amino acid
YIFPNNGGNG

SEQ ID NO: 35 - protein, 11 aa, Mus sp.
  Clone M8 IL38-C VH CDR3 amino acid
ARGGYDAGFVY

SEQ ID NO: 36 - DNA, 370 bp, Mus sp.
  Clone M27 IL38-C HC nucleic acid
gaggtgcagcttgttgagtctggtggaagattggtacagcctaaagggtcattgaaactctcatgtgcagcctctggattcaccttcaatacctatgccatgtactggatccgccaggctccaggaaagggtttggaatgggttgctcgcataagaactaaaagtaataattttgcaacatattatgccgattcagtgaaagacagattcaccatctccagagatgattcacaaaacatgctctatctgcaaatgaacaacctgaaaactgaggacacagccatgtattactgtgtgctcggatttggatggcccacttactatactctggactactggggtcaaggaacctcagtcaccgtctcctcag

SEQ ID NO: 37 - protein, 123 aa, Mus sp.
  Clone M27 IL38-C VH amino acid
EVQLVESGGRLVQPKGSLKLSCAASGFTFNTYAMYWIRQAPGKGLEWVARIRTKSNNFATYYADSVKDRFTISRDDSQNMLYLQMNNLKTEDTAMYYCVLGFGWPTYYTLDYWGQGTSVTVSS

SEQ ID NO: 38 - protein, 13 aa, Mus sp.
  Clone M27 IL38-C VH CDR1 amino acid
AASGFTFNTYAMY

SEQ ID NO: 39 - protein, 12 aa, Mus sp.
  Clone M27 IL38-C VH CDR2 amino acid
RIRTKSNNFATY

SEQ ID NO: 40 - protein, 14 aa, Mus sp.
  Clone M27 IL38-C VH CDR3 amino acid
VLGFGWPTYYTLDY

SEQ ID NO: 41 - DNA, 358 bp, Mus sp.
  Clone M2 IL38-N VH nucleic acid
caggtccaactgcagcagcctggkgctgagcttgtgaagcctggggcctcagtgaagctgtcctgcaaggcttctggctacactttcaccagctactggataaactgggtgaagcagaggcctggacaaggccttgagtggattggaaatatttatcctgttagtagtaatactaagtacaatgagaagttcaagagtaaggccacactgactgtagacacatcctccagcacagcctacatgcagctcagcagcctgacatctgacgactctgcggtctattattgtgcaagaggggggtactacggttatgctatggactactggggtcaaggaacctcagtcaccgtctcctcag

SEQ ID NO: 42 - protein, 119 aa, Mus sp.
  Clone M2 IL38-N VH amino acid
QVQLQQPGAELVKPGASVKLSCKASGYTFTSYWINWVKQRPGQGLEWIGNIYPVSSNTKYNEKFKSKATLTVDTSSSTAYMQLSSLTSDDSAVYYCARGGYYGYAMDYWGQGTSVTVSS

SEQ ID NO: 43 - protein, 13 aa, Mus sp.
  Clone M2 IL38-N VH CDR1 amino acid
KASGYTFTSYWIN

SEQ ID NO: 44 - protein, 10 aa, Mus sp.
  Clone M2 IL38-N VH CDR2 amino acid
NIYPVSSNTK

SEQ ID NO: 45 - protein, 12 aa, Mus sp.
  Clone M2 IL38-N VH CDR3 amino acid
ARGGYYGYAMDY

SEQ ID NO: 46 - DNA, 340 bp, Mus sp.
  Clone M8 IL38-N VH nucleic acid
gaggtccagctgcagcagtctggacctgagctggtgaagcctggggcttcagtgaagatatcctgcaaggcttctggttactcattcactggctactacatgcactgggtgaagcaaagtcctgaaaagagccttgagtggattggagtgattaatcctaacactggtggtattacctacaaccagaagttcaaggccaaggccacattgaatgtagacaaatcctccagcacagcctacatgcagctcaagagcctgacatctgaggactctgcagtctattactgtgcaagatcgatgggagtttggggccaagggactctggtcactgtctctgcag

SEQ ID NO: 47 - protein, 113 aa, Mus sp.
  Clone M8 IL38-N VH amino acid
EVQLQQSGPELVKPGASVKISCKASGYSFTGYYMHWVKQSPEKSLEWIGVINPNTGGITYNQKFKAKATLNVDKSSSTAYMQLKSLTSEDSAVYYCARSMGVWGQGTLVTVSA

SEQ ID NO: 48 - protein, 13 aa, Mus sp.
  Clone M8 IL38-N VH CDR1 amino acid
KASGYSFTGYYMH

SEQ ID NO: 49 - protein, 10 aa, Mus sp.
  Clone M8 IL38-N VH CDR2 amino acid
VINPNTGGIT

SEQ ID NO: 50 - protein, 6 aa, Mus sp.
  Clone M8 IL38-N VH CDR3 amino acid
ARSMGV

SEQ ID NO: 51 - DNA, 349 bp, Mus sp.
  Clone M12 IL38-N VH nucleic acid
caggttcaactgcagcagtctggacctgagctggtgaagcctggggcctcagtgaagatttcctgcaaagcttctggctacgcattcagtagctactggatgaactgggtgaagcagaggcctggaaagggtcttgagtggattggacggatttatcctggagatggaaatactaagtacaatgggatgttcaagggcaaggccacactgactgcagacaaatcctccagcacagcctacatgcaactcagcagcctgacatctgaggactctgcggtcttcttctgtgcaagaggggcacgtggggaggactactggggtcaaggaacctcagtcaccgtctcctcag

SEQ ID NO: 52 - protein, 116 aa, Mus sp.
  Clone M12 IL38-N VH amino acid
QVQLQQSGPELVKPGASVKISCKASGYAFSSYWMNWVKQRPGKGLEWIGRIYPGDGNTKYNGMFKGKATLTADKSSSTAYMQLSSLTSEDSAVFFCARGARGEDYWGQGTSVTVSS

SEQ ID NO: 53 - protein, 13 aa, Mus sp.
  Clone M12 IL38-N VH CDR1 amino acid
KASGYAFSSYWMN

SEQ ID NO: 54 - protein, 10 aa, Mus sp.
  Clone M12 IL38-N VH CDR2 amino acid
RIYPGDGNTK

SEQ ID NO: 55 - protein, 9 aa, Mus sp.
  Clone M12 IL38-N VH CDR3 amino acid
ARGARGEDY

SEQ ID NO: 56 - DNA, 337 bp, Mus sp.
  Clone M3 IL38-C VL nucleic acid
gatgttgtgatgacccaatctccactctccctgcctgtcagtcttggagatcaagcctccatctctcgcagatttagtcagagccttgtacacagtcatgaaaacacctttttacattggtacgtgcagaagccaggccagtctccaaagctcctgatttacagagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaac

SEQ ID NO: 57 - protein, 112 aa, Mus sp.
  Clone M3 IL38-C VL amino acid
DVVMTQSPLSLPVSLGDQASISCRSSQSLVHSHENTFLHWYLQKPGQSPKLLIYRVSNRFSGVPDRFSGSGSGTDFTLKISRVEAEDLGVYFCSQSTHVPLTFGAGTKLELK

SEQ ID NO: 58 - protein, 16 aa, Mus sp.
  Clone M3 IL38-C VL CDR1 amino acid
RSSQSLVHSHENTFLH

SEQ ID NO: 59 - protein, 8 aa, Mus sp.
  Clone M3 IL38-C VL CDR2 amino acid
YRVSNRFS

SEQ ID NO: 60 - protein, 9 aa, Mus sp.
  Clone M3 IL38-C VL CDR3 amino acid
SQSTHVPLT

SEQ ID NO: 61 - DNA, 337 bp, Mus sp.
  Clone M8 IL38-C VL nucleic acid
gacattgtgatgtcacagtctccatcctccctggctgtgtcagcaggagagaaggtcactatgagctgcaaatccagtcagagtctgttcaacagtggagcccgaaagaacttcttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttattatctgatcacgttcggtgctgggaccaagctggagctgaaac

SEQ ID NO: 62 - protein, 112 aa, Mus sp.
  Clone M8 IL38-C VL amino acid
DIVMSQSPSSLAVSAGEKVTMSCKSSQSLFNSGARKNFLAWYQQKPGQSPKLLIYWASTRESGVPDRFTGSGSGTDFTLTISSVQAEDLAVYYCKQSYYLITFGAGTKLELK

SEQ ID NO: 63 - protein, 17 aa, Mus sp.
  Clone M8 IL38-C VL CDR1 amino acid
KSSQSLFNSGARKNFLA

SEQ ID NO: 64 - protein, 8 aa, Mus sp.
  Clone M8 IL38-C VL CDR2 amino acid
YWASTRES

SEQ ID NO: 65 - protein, 8 aa, Mus sp.
  Clone M8 IL38-C VL CDR3 amino acid
KQSYYLIT

SEQ ID NO: 66 - DNA, 325 bp, Mus sp.
  Clone M27 IL38-C VL nucleic acid
caaattgttctcacccagtctccagcaatcatgtctgcctctccaggggaaaaggtcaccatgccctgcagtgccatgtcaagtgtcagttccaggtacttgcactggaaccagcagaagtcaggagcctcccccaaactctggatctatggcgcatccaacctggcttctggagtccctgctcggttcagtggcagtgggtctgggacctcctactctctcacaatcatcagcgtggaggatgaagatgctgccacctattactgccagcagtatcatagtggagcgctcacgttcggaggggggaccaagctggagatgaaac

SEQ ID NO: 67 - protein, 108 aa, Mus sp.
  Clone M27 IL38-C VL amino acid
QIVLTQSPAIMSASPGEKVTMPCSAMSSVSSRYLHWNQQKSGASPKLWIYGASNLASGVPARFSGSGSGTSYSLTIISVEDEDAATYYCQQYHSGALTFGGGTKLEMK

SEQ ID NO: 68 - protein, 12 aa, Mus sp.
  Clone M27 IL38-C VL CDR1 amino acid
SAMSSVSSRYLH

SEQ ID NO: 69 - protein, 8 aa, Mus sp.
  Clone M27 IL38-C VL CDR2 amino acid
YGASNLAS

SEQ ID NO: 70 - protein, 9 aa, Mus sp.
  Clone M27 IL38-C VL CDR3 amino acid
QQYHSGALT

SEQ ID NO: 71 - DNA, 322 bp, Mus sp.
  Clone M2 IL38-N VL nucleic acid
gacatccagatgactcagtctccagcctccctatctgcatctgtgggagaaactgtcaccctcacatgtcgaccaagtgggaatgttcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccattcacgttcggctcggggacaaagttggaaataaaac

SEQ ID NO: 72 - protein, 107 aa, Mus sp.
  Clone M2 IL38-N VL amino acid
DIQMTQSPASLSASVGETVTLTCRPSGNVHNYLAWYQQKQGKSPQLLVYNAKTLADGVPSRFSGSGSGTQYSLKINSLQPEDFGSYYCQHFWSTPFTFGSGTKLEIK

SEQ ID NO: 73 - protein, 11 aa, Mus sp.
  Clone M2 IL38-N VL CDR1 amino acid
RPSGNVHNYLA

SEQ ID NO: 74 - protein, 8 aa, Mus sp.
  Clone M2 IL38-N VL CDR2 amino acid
YNAKTLAD

SEQ ID NO: 75 - protein, 9 aa, Mus sp.
  Clone M2 IL38-N VL CDR3 amino acid
QHFWSTPFT

SEQ ID NO: 76 - DNA, 337 bp, Mus sp.
  Clone M8 IL38-N VL nucleic acid
gatattgtgatgactcaggctgcaccctctgtatctgtcactcctggagagtcagtatccatctcctccaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaatatctagaatatcctttcacgttcggctcggggacaaagctggaaataaaac

SEQ ID NO: 77 - protein, 112 aa, Mus sp.
  Clone M8 IL38-N VL amino acid
DIVMTQAAPSVSVTPGESVSISCRSSKSLLHSNGNTYLYWFLQRPGQSPQLLIYRMSNLASGVPDRFSGSGSGTAFTLRISRVEAEDVGVYYCMQYLEYPFTFGSGTKLEIK

SEQ ID NO: 78 - protein, 16 aa, Mus sp.
  Clone M8 IL38-N VL CDR1 amino acid
RSSKSLLHSNGNTYLY

SEQ ID NO: 79 - protein, 8 aa, Mus sp.
  Clone M8 IL38-N VL CDR2 amino acid
YRMSNLAS

SEQ ID NO: 80 - protein, 9 aa, Mus sp.
  Clone M8 IL38-N VL CDR3 amino acid
MQYLEYPFT

SEQ ID NO: 81 - DNA, 325 bp, Mus sp.
  Clone M12 IL38-N VL nucleic acid
gaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacggtgattttccactcacgttcggaggggggaccaagctggaaataaaac

SEQ ID NO: 82 - protein, 108 aa, Mus sp.
  Clone M12 IL38-N VL amino acid
ENVLTQSPAIMSASPGEKVTMTCRASSSVSSSYLHWYQQKSGASPKLWIYSTSNLASGVPARFSGSGSGTSYSLTISSVEAEDAATYYCQQYGDFPLTFGGGTKLEIK

SEQ ID NO: 83 - protein, 12 aa, Mus sp.
  Clone M12 IL38-N VL CDR1 amino acid
RASSSVSSSYLH

SEQ ID NO: 84 - protein, 8 aa, Mus sp.
  Clone M12 IL38-N VL CDR2 amino acid
YSTSNLAS

SEQ ID NO: 85 - protein, 9 aa, Mus sp.
  Clone M12 IL38-N VL CDR3 amino acid
QQYGDFPLT

(19) United States
(12) Patent Application Publication
(10) Pub. No.: US 2022/0275077 A1
(43) Pub. Date: Sep. 1, 2022

(54) IL-38-SPECIFIC ANTIBODIES

(71) Applicant: Immunome, Inc., Exton, PA (US)

(72) Inventors: Matthew K. ROBINSON, Blue Bell, PA (US); Karen LUNDGREN, Solomons, MD (US); Ben HARMAN, Downingtown, PA (US); Pavel A. NIKITIN, Downingtown,, PA (US); John P. DOWLING, Willow Grove, PA (US)

(21) Appl. No.: 17/631,102

(22) PCT Filed: Jul. 30, 2020
(86) PCT No.: PCT/US2020/044260

§ 371 Date: Jan. 28, 2022

Related U.S. Application Data
(60) Provisional application No. 62/880,265, filed on Jul. 30, 2019.

(51) Int. Cl. C07K 16/24; A61K 39/395; A61P 35/00; A61P 35/04

(52) U.S. Cl. CPC C07K 16/244; A61K 39/3955; A61P 35/00; A61K 2039/505; C07K 2317/622; C07K 2317/526; A61P 35/04

(57) ABSTRACT

This invention relates to antibodies specific for human interleukin-38 (IL-38), including isolated antibodies, or antigen-binding fragments thereof. The antibodies, or antigen-binding fragments thereof, described here partially or fully block, inhibit, or neutralize a biological activity of IL 38. Methods described here relate to inhibiting tumor growth or metastasis in an individual afflicted by tumor growth and/or metastasis.

### CROSS REFERENCE TO RELATED APPLICATIONS

This application claims priority to U.S. Application No. 62/880,265, filed on Jul. 30, 2019, which is incorporated by reference herein in its entirety.

### FIELD OF THE INVENTION

The field of this invention relates to therapeutically useful Interleukin-38 (IL-38)-specific molecules

### BACKGROUND

The human adaptive immune system responds through both cellular (T cell) and humoral (B cell) processes. The humoral response results in selection and clonal amplification of B cells that express surface bound immunoglobulin (Ig) molecules capable of binding to antigens. The processes of somatic hypermutation and class switching take place concordant with the clonal amplification. Together these processes lead to secreted antibodies that have been affinity matured against a target antigen and contain a constant domain belonging to one of the five general classes, also called isotypes, (M, D, A, G, or E). Each class of antibody (IgM, IgD, IgA, IgG, and IgE) interact in distinct ways with the cellular immune system. Hallmarks of antibodies that have been affinity matured against a target antigen can include: 1) nucleotide, and subsequent amino acid, changes relative to the germline gene, 2) high binding affinity for the target antigen, 3) binding selectivity for the target antigen as compared to other proteins.

It is well understood that oncology patients can mount an immune response against tumor antigens. Those antigens can result either from genetic changes within the tumor that lead to mutated proteins or aberrant presentation, of otherwise normal, proteins to the immune system. Aberrant presentation may occur through processes that include, but are not limited to, ectopic expression of neonatal proteins, over-expression of proteins to a high level, mis-localization of intracellular proteins to the cell surface, or lysis of cells. Aberrant glycosylation of proteins, which may occur because of changes in the expression of enzymes, such as, but not limited to, glycosyltransferases, can also result in generation of non-self antigens that are recognized by the humoral immune system.

Antibodies, which bind selectively to disease-related proteins, including proteins related to cancer, have proven successful at modulating the functions of their target proteins in ways that lead to therapeutic efficacy. The ability of the human immune system to mount antibody responses against mutated, or otherwise aberrant, proteins suggests that patients' immune responses may include antibodies that are capable of recognizing, and modulating the function of, critical tumor-drivers.

The tumor microenvironment is essential for tumor cells to evade detection and elimination by the immune system. This is achieved through several mechanisms including the recruitment of suppressive immune cells, expression of immune checkpoint molecules like PD-1, and the presence of immunosuppressive cytokines. Therefore, some immune-oncology therapies aim to target key molecules that are responsible for the immunosuppressive barriers within the tumor microenvironment. Successful immune-oncology therapies can lead to the infiltration of cytotoxic T cells and NK cells as well as an upregulation of Th1 cytokines and the induction of a successful anti-tumor response.

The IL-1 family of cytokines, of which IL-38 is a member, elicit downstream signaling events by binding to, and forming complexes with, membrane bound receptors. An example of an IL-1 receptor complex is represented in FIG. 1. As with other cytokines, such as Tumor Necrosis Factor Alpha (TNFa), binding of antibodies to one, or more, epitopes on the cytokines can be predicted to interfere with the ability of the cytokine to form complexes with their cognate receptors (Hu et al., J B C, 2013). Residues of IL-1, represented in spheres in FIG. 1, are predicted to form an interaction face between IL-1 and the receptors Interleukin-1 Receptor 2 (IL-1R2) and Interleukin-1 Receptor Accessory Protein (IL-1RAP). Residues within this predicted receptor binding region of IL-1 may comprise epitopes to which antibodies can bind. Binding of antibodies to those epitopes would be predicted to block binding of IL-1 to its cognate receptors and antagonize, or inhibit, the cytokine's biological function. The homology between IL-1 and IL-38 suggests that a corresponding epitope, or epitopes, exist on IL-38. Binding of antibodies to that epitope, or epitopes, would be predicted to antagonize, or inhibit, the function of IL-38.

The IL-1 family of cytokines plays an important role in regulating inflammation during tumor development as well as during treatment (Baker et al., 2019). While some IL-1 family members promote inflammation, others can suppress it. IL-38 is an antagonist that blocks signaling through IL-1 family receptors, including IL-36R and IL1RALP1, and serves as an immune checkpoint by dampening inflammation (Veerdonk et al., 2017). In particular, IL-38 is shown to reduce the production of inflammatory cytokines, which can play key roles in an effective anti-tumor response. In addition, IL-38 secreted from apoptotic tumor cells can suppress macrophage and T cell responses in vitro (Mora et al., 2016). Similarly, IL-38 expression has been shown to correlate with poor prognosis in patients with lung adenocarcinoma as well as correlate with PDL1 expression (Takada et al., 2017). However, conflicting evidence has been reported in non small cell lung cancer, where lower expression of IL-38 correlates with poor prognosis (Wang et al., 2018). IL-38 may serve as an immunosuppressive cytokine within the tumor microenvironment and thus blocking IL-38 signaling may trigger an effective anti-tumor response (FIG. 2).

### SUMMARY OF THE INVENTION

This invention relates to antibodies specific for human interleukin-38 (IL-38), including isolated antibodies, or antigen-binding fragments thereof, which contain at least 3, at least 4, at least 5, at least 6, at least 7, or at least 8 optionally contiguous amino acids of at least one complementary-determining region (CDR) contained within the variable heavy chain (VH) amino acid sequences of: SEQ ID NO: 22, SEQ ID NO: 27, SEQ ID NO: 32, SEQ ID NO: 37, SEQ ID NO: 42, SEQ ID NO: 47, SEQ ID NO: 52, SEQ ID NO: 2, or SEQ ID NO: 7; and/or contained within the variable light chain (VH) amino acid sequences of: SEQ ID NO: 57, SEQ ID NO: 62, SEQ ID NO: 67, SEQ ID NO: 72, SEQ ID NO: 77, SEQ ID NO: 82, SEQ ID NO: 4, or SEQ ID NO: 9.

More particularly, the CDRs of an antibody of the invention, or antigen-binding fragment may contain at least 3, at least 4, at least 5, at least 6, at least 7, or at least 8 optionally contiguous amino acids of: the VH CDR1 amino acid sequences of SEQ ID NOS: 23, 28, 33, 38, 43, 48, 53, or 15;

the VH CDR2 amino acid sequences of SEQ ID NOS: 24, 29, 34, 39, 44, 49, 54, or 16;
the VH CDR3 amino acid sequences of SEQ ID NOS: 25, 30, 35, 40, 45, 50, 55, or 17;
the VL CDR1 amino acid sequences of SEQ ID NOS: 58, 63, 68, 73, 78, 83, or 18;
the VL CDR2 amino acid sequences of SEQ ID NOS: 59, 64, 69, 74, 79, 84, or 19; and/or
the VL CDR3 amino acid sequences of SEQ ID NOS: 60, 65, 70, 75, 80, 85, or 20.

Antibodies of the invention, or antigen-binding fragments thereof, partially or fully blocks, inhibits, or neutralizes a biological activity of IL 38. Accordingly, methods of the invention include methods of inhibiting tumor growth or metastasis in an individual afflicted by tumor growth and/or metastasis by administering a therapeutically effective amount of a composition comprising the antibody or an antigen-binding fragment thereof, to the individual for treatment in which the antibody of the invention, or an antigen-binding fragment thereof, partially or fully blocks, inhibits, or neutralizes a biological activity of IL-38 that promotes or sustains tumor growth and/or metastasis.

### BRIEF DESCRIPTION OF THE FIGURES

FIG. 1 is a representation of a co-crystal structure of an Interleukin-1 (IL-1) receptor complex (RCSB PDB file number 3040) visualized using PyMol. The co-crystal comprises IL-1Beta, Interleukin-1 receptor type 2 (IL-1R2), and Interleukin-1 receptor accessory protein (IL-1RAP). IL-1R2 and IL-1RAP are depicted in black and light gray cartoons, respectively. IL-1Beta is depicted in medium gray ribbon. Residues of IL-1Beta depicted in spheres are those predicted to be within 4 Angstroms of either IL-1R2 or IL-1RAP.

FIG. 2 is a cartoon depicting how blocking IL-38 function may elicit an inflammatory response that could lead to an anti-tumor response.

FIG. 3. is a graph showing binding pattern of PR087-29B5 hybridoma-produced antibodies in a LICOR-based screen.

FIG. 4 is a graph showing selective, dose-dependent binding of PR087-29B5 to recombinant human IL-38 in a dot blot format.

FIG. 5 is a graph showing binding of IMM20130 to various cell lines by flow cytometry

FIG. 6. is a graph showing IL-38 concentration in conditioned media of cancer cell lines cultured under apoptotic conditions for varying timepoints.

FIG. 7 is a set of graphs showing RNA expression analyses based on data from the TCGA database comparing IL-38 expression levels with immune cell lineage-specific markers in prostate adenocarcinoma (PRAD), colorectal adenocarcinoma (COAD), lung adenocarcinoma (LUAD), skin cutaneous melanoma (SKCM), uterine corpus endometrial carcinoma (UCEC), head & neck squamous cell carcinoma (HNSC) and pancreatic adenocarcinoma (PAAD).

FIG. 8 is a graph of IL-6 and TNFα expression in conditioned media from LPS-stimulated THP-1 macrophages.

FIG. 9 is a graph of markers that were downregulated in LPS-stimulated macrophages when treated with IL-38.

FIG. 10 is a graph of the phosphorylation status of Jnk and STAT3 in THP-1 macrophages stimulated with LPS for indicated timepoints.

FIG. 11 is a graph showing IL-6 production of LPS-stimulated THP-1 cells treated with various combinations of IL-38, IMM20130, and an isotype control.

FIG. 12 is a graph showing IL-6 production of LPS-stimulated THP-1 cells treated with various combinations of IL-38 and anti-IL-38 polyclonal antibodies.

FIG. 13 is a graph showing binding of monoclonal supernatants to recombinant IL-38.

FIG. 14 is a graph showing percent rescue of monoclonal supernatants, defined by their ability to restore IL-6 production in IL-38-treated, LPS-stimulated cells.

FIG. 15 is a graph showing binding kinetics of a selected anti-human IL-38 antibody

FIG. 16A is a graph showing a dose response of lead candidate antibodies tested for their ability to restore IL-6 production in IL-38-treated, LPS-stimulated antibodies.

FIG. 16B is a graph showing a dose response of lead candidate antibodies tested for their ability to restore and GM-CSF production in IL-38-treated, LPS-stimulated antibodies.

FIG. 17 is a graph of the plasma levels of lead candidate antibodies overtime in C57BL/6 mice dosed 10 mg/kg both i.v. and i.p.

FIG. 18 is a growth curve of B16.F10 tumors implanted in C57BL/6 mice treated with CD1-M3, paclitaxel, or a combination.

FIG. 19 is a graph characterizing the tumor infiltrating myeloid and lymphocyte populations by flow cytometry in B16.F10 tumors treated with CD1-M3.

FIG. 20 is a growth curve of MMTV-PyMT tumors implanted in FVB mice treated with CD1-M3 and a graph showing IL-6 levels within these tumors.

FIG. 21 is a growth curve of A549 tumors implanted in scid mice treated with CD1-M3, CD1-M8, CD1-M26, and NZB-M8.

FIG. 22 is a graph characterizing the tumor infiltrating myeloid populations by flow cytometry in A549 tumors treated with CD1-M3, CD1-M8, CD1-M26, and NZB-M8.

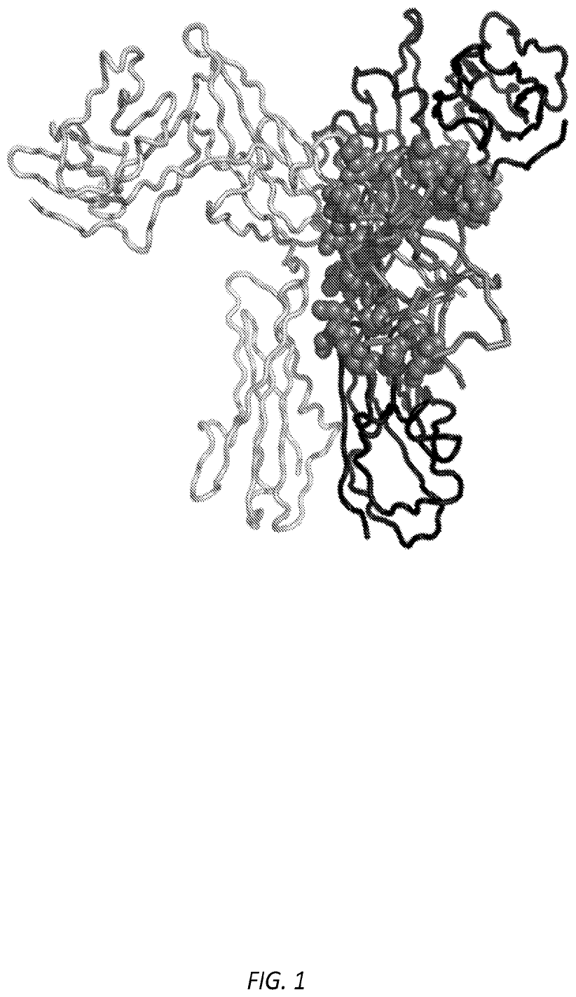

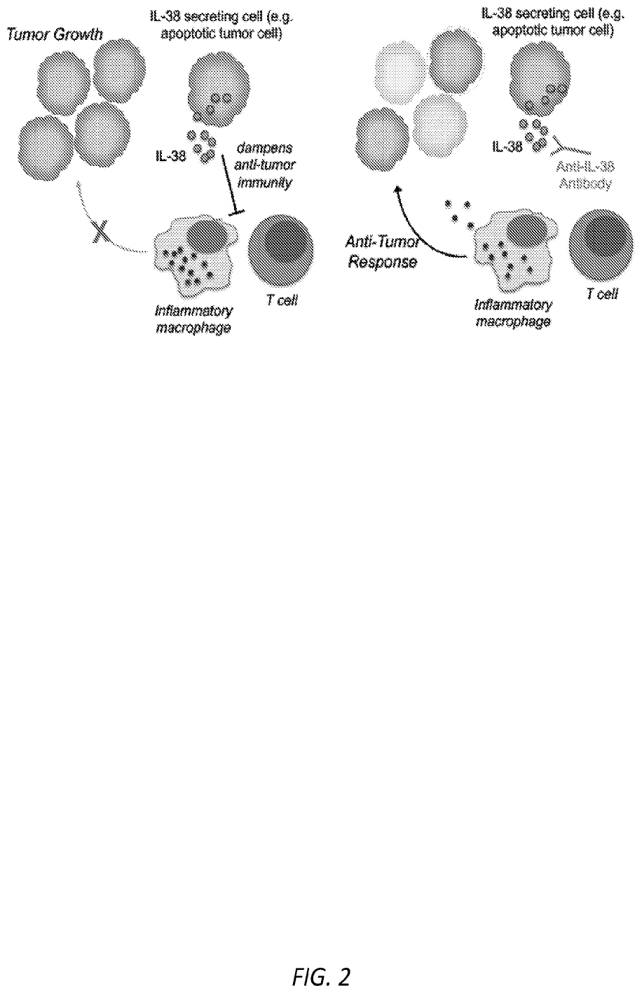

### DETAILED DESCRIPTION

The invention described herein relates to antibodies that specifically bind to Interleukin-38 (IL 38). Accordingly, the invention includes compositions of antibodies specific for IL-38, methods for using antibodies specific for IL-38, and methods for preparing and formulating antibodies specific for IL-38. Methods of using antibodies of the invention may include methods of treating individuals in need thereof. Accordingly, methods of using antibodies of the invention in methods of treatment may include methods of administering antibody compositions of the invention. Methods of the invention may also include using the antibodies in in vivo and in vitro diagnostic methods. Diagnostic methods of the invention may be included as a step in a multi-step method of treatment.

An antibody according to the invention may be an intact immunoglobulin, or a variant of an immunoglobulin, or a portion of an immunoglobulin. A naturally occurring immunoglobulin has two heavy (H) chains and two light (L) chains, each of which, contains a constant region and a variable region, and are interconnected by disulfide bonds. There are two types of light chains, which are termed lambda (“A”) and kappa (“K”). There are five main heavy chain classes, also known as isotypes, which determine functional activity of an antibody molecule: IgM, IgD, IgG, IgA and IgE. In addition to its variable domain, an IgA, IgD, or IgG heavy chain has three constant domains (CH1, CH2, CH3). IgM and IgE heavy chains have four constant domains (CH1, CH2, CH3, CH4).

Light and heavy chain variable regions contain “framework” regions interrupted by three hypervariable regions, called complementarity-determining regions (“CDRs”). The CDRs are primarily responsible for binding to an epitope of an antigen. The sequences of the framework regions of different light or heavy chains are relatively conserved within a species, and serve to position and align the CDRs in three-dimensional space. The three CDRs of each chain are typically referred to as CDR1, CDR2, and CDR3, numbered sequentially starting from the N-terminus, and are often identified by the chain in which the particular CDR is located. Accordingly, heavy chain CDRs are designated H-CDR1, H-CDR2, and H-CDR3; likewise, light chain CDRs are designated L-CDR1, L-CDR2, and L-CDR3. An antigen-binding fragment that is composed of one constant and one variable domain of each of the heavy and the light chain is referred to as an Fab fragment. An F(ab)′₂ fragment contains two Fab fragments, and can be generated by cleaving an immunoglobulin molecule below its hinge region.

The amino acid sequence of an antibody of the invention can also contain a variant of one or more of the amino acid sequences of: SEQ. ID. NOS. 2, 4, 7, 10, 12, 14-20, 22-25, 27-30, 32-35, 37-40, 42-45, 47-50, 52-55, 57-60, 62-65, 67-70, 72-75, 77-80, and 82-85; or one or more of the amino acid sequences encoded by SEQ. ID. NOS. 1, 3, 5, 6, 8, 9, 11, 13, 21, 26, 31, 36, 41, 46, 51, 56, 61, 66, 71, 76, and 81. Antibody variants typically contain amino acid sequence modifications, and can be made for any reason, including, for example, to improve specificity, affinity, or stability (i.e., half-life). Examples of antibody variants of the invention include, but are not limited to, fragments of antibodies, amino acid substitutions, amino acid deletions, chimeric antibodies, and any combination of the foregoing.

A variant antibody of the invention that contains one or more amino acid substitutions generally contain no more than 15, no more than 12, no more than 10, no more than 9, no more than 8, no more than 7, no more than 6, no more than 5, no more than 4, no more than 3, no more than 2 or no more than 1 conservative amino acid substitution relative to an amino acid sequence of SEQ. ID. NOS. 2, 4, 7, 10, 12, 14-20, 22-25, 27-30, 32-35, 37-40, 42-45, 47-50, 52-55, 57-60, 62-65, 67-70, 72-75, 77-80, and 82-85; or one or more of the amino acid sequences encoded by SEQ. ID. NOS. 1, 3, 5, 6, 8, 9, 11, 13, 21, 26, 31, 36, 41, 46, 51, 56, 61, 66, 71, 76, and 81; and/or no more than 5, no more than 4, no more than 3, or no more than 2 non-conservative amino acid substitutions, or no more than 1 non-conservative amino acid substitution, relative to an amino acid sequence of: SEQ. ID. NOS. 2, 4, 7, 10, 12, 14-20, 22-25, 27-30, 32-35, 37-40, 42-45, 47-50, 52-55, 57-60, 62-65, 67-70, 72-75, 77-80, and 82-85; or one or more of the amino acid sequences encoded by SEQ. ID. NOS. 1, 3, 5, 6, 8, 9, 11, 13, 21, 26, 31, 36, 41, 46, 51, 56, 61, 66, 71, 76, and 81.

A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). Variant antibodies of the invention may include amino acid substitutions with amino acid analogs as well as amino acids, as described herein. An antibody of the invention can contain one or more amino acid sequences that share at least 85% sequence identity with the amino acid sequence of one or more of the amino acid sequences of SEQ. ID. NOS. 2, 4, 7, 10, 12, 14-20, 22-25, 27-30, 32-35, 37-40, 42-45, 47-50, 52-55, 57-60, 62-65, 67-70, 72-75, 77-80, and 82-85; or one or more of the amino acid sequences encoded by SEQ. ID. NOS. 1, 3, 5, 6, 8, 9, 11, 13, 21, 26, 31, 36, 41, 46, 51, 56, 61, 66, 71, 76, and 81. Accordingly, an antibody of the invention can have an amino acid sequence that shares at least 85%, 86%, 87%, 88%, 89% 90% 91%, 92%, 93%, 94%, 95%, 96%, 97%, 98%, or 99% sequence identity with the amino acid sequence of one or more of the amino acid sequences of SEQ. ID. NOS. 2, 4, 7, 10, 12, 14-20, 22-25, 27-30, 32-35, 37-40, 42-45, 47-50, 52-55, 57-60, 62-65, 67-70, 72-75, 77-80, and 82-85; or one or more of the amino acid sequences encoded by SEQ. ID. NOS. 1, 3, 5, 6, 8, 9, 11, 13, 21, 26, 31, 36, 41, 46, 51, 56, 61, 66, 71, 76, and 81. As used herein, the term “sequence identity” refers to the similarity between two, or more, amino acid sequences. Sequence identity is typically measured in terms of percentage identity (or similarity or homology) between amino acid sequences; the higher the percentage, the more similar to each other are the compared sequences.

The amino acid sequence of the V_{H} framework region of some antibodies of the invention may contain a sequence, or fragment thereof, of SEQ ID NOs: 22, 27, 32, 37, 42, 47, 52, 2, or 7. Similarly, the amino acid sequences of the V_{L} framework region of the same, or different, antibodies may contain a sequence, or antigen-binding fragment thereof, of SEQ ID NOs: 57, 62, 67, 72, 77, 82, 4, or 10.

An antibody of the invention contains at least 1, at least 2, at least 3, at least 4, at least five, or at least 6 CDRs. The amino acid sequences of CDRs in antibodies of the invention can be determined by known methods, and definitions of CDRs, in the art, including, for example: the ImMunoGeneTics database (“IMGT”) numbering system, (Lefranc, M.-P. et al., Nucleic Acids Research, 27, 209-212 (1999)); the CDR definition(s) described by North, B. et al. (A new clustering of antibody CDR loop conformations, J Mol Biol (2011)); CDR definition(s) Kabat et al., Sequences of Proteins of Immunological Interest, 5th Ed. Public Health Service, National Institutes of Health, Bethesda, Md. (1991); CDR definition(s) Chothia et al., J. Mol. Biol. 196:901-917 (1987); and CDR definition(s) MacCallum et al., J. Mol. Biol. 262:732-745 (1996).

One or more of the CDRs of some antibodies, or antigen-binding fragments, of the invention contain at least 3, at least 4, at least 5, at least 6, at least 7, or at least 8, at least 9, at least 10, at least 11, or at least 12 contiguous amino acids of: the VH CDR1 are selected from the amino acid sequences of SEQ ID NOS: 23, 28, 33, 38, 43, 48, 53, or 15; the VH CDR2 are selected from the amino acid sequences of SEQ ID NOS: 24, 29, 34, 39, 44, 49, 54, or 16; the VH CDR3 are selected from the amino acid sequences of SEQ ID NOS: 25, 30, 35, 40, 45, 50, 55, or 17; the VL CDR1 are selected from the amino acid sequences of SEQ ID NOS: 58, 63, 68, 73, 78, 83, or 18; the VL CDR2 are selected from the amino acid sequences of SEQ ID NOS: 59, 64, 69, 74, 79, 84, or 19; and the VL CDR3 are selected from the amino acid sequences of SEQ ID NOS: 60, 65, 70, 75, 80, 85, or 20.

In some embodiments, an antibody of the invention contains at least one, at least two, at least three, at least four, at least five, or at least six of: a VH CDR1 of SEQ ID NO:23, a VH CDR2 of SEQ ID NO: 24, a VH CDR3 of SEQ ID NO: 25, a VL CDR1 of SEQ ID NO: 58, a VL CDR2 of SEQ ID NO:59, and a VL CDR3 of SEQ ID NO: 60.

In other embodiments, an antibody of the invention contains at least one, at least two, at least three, at least four, at least five, or at least six of: a VH CDR1 of SEQ ID NO:28, a VH CDR2 of SEQ ID NO: 29, a VH CDR3 of SEQ ID NO: 30, a VL CDR1 of SEQ ID NO: 58, a VL CDR2 of SEQ ID NO:59, and a VL CDR3 of SEQ ID NO: 60.

In other embodiments, an antibody of the invention contains at least four, at least five, or at least six of: a VH CDR1 of SEQ ID NO: 33, a VH CDR2 of SEQ ID NO: 34, a VH CDR3 of SEQ ID NO: 35, a VL CDR1 of SEQ ID NO: 63, a VL CDR2 of SEQ ID NO: 64, and a VL CDR3 of SEQ ID NO: 65.

In other embodiments, an antibody of the invention contains at least four, at least five, or at least six of: a VH CDR1 of SEQ ID NO: 38, a VH CDR2 of SEQ ID NO: 39, a VH CDR3 of SEQ ID NO: 40, a VL CDR1 of SEQ ID NO: 68, a VL CDR2 of SEQ ID NO: 69, and a VL CDR3 of SEQ ID NO: 70.

In other embodiments, an antibody of the invention contains at least four, at least five, or at least six of: a VH CDR1 of SEQ ID NO: 43, a VH CDR2 of SEQ ID NO: 44, a VH CDR3 of SEQ ID NO: 45, a VL CDR1 of SEQ ID NO: 73, a VL CDR2 of SEQ ID NO: 74, and a VL CDR3 of SEQ ID NO: 75.

In other embodiments, an antibody of the invention contains at least four, at least five, or at least six of: a VH CDR1 of SEQ ID NO: 48, a VH CDR2 of SEQ ID NO: 49, a VH CDR3 of SEQ ID NO: 50, a VL CDR1 of SEQ ID NO: 78, a VL CDR2 of SEQ ID NO: 79, and a VL CDR3 of SEQ ID NO: 80.

In other embodiments, an antibody of the invention contains at least four, at least five, or at least six of: a VH CDR1 of SEQ ID NO: 53, a VH CDR2 of SEQ ID NO: 54, a VH CDR3 of SEQ ID NO: 55, a VL CDR1 of SEQ ID NO: 83, a VL CDR2 of SEQ ID NO: 84, and a VL CDR3 of SEQ ID NO: 85.

In other embodiments, an antibody of the invention contains at least four, at least five, or at least six of: a VH CDR1 of SEQ ID NO: 15, a VH CDR2 of SEQ ID NO: 16, a VH CDR3 of SEQ ID NO: 17, a VL CDR1 of SEQ ID NO: 18, a VL CDR2 of SEQ ID NO: 19, and a VL CDR3 of SEQ ID NO: 20.

Antibodies according to the invention are monoclonal antibodies, meaning an antibody is produced by a single clonal B-lymphocyte population, a clonal hybridoma cell population, or a clonal population of cells into which the genes of a single antibody, or portions thereof, have been transfected. Monoclonal antibodies are produced by methods known to those of skill in the art, for instance by making hybrid antibody-forming cells from a fusion of myeloma cells with immune lymphocyte cells.

Monoclonal antibodies according to the invention are also typically humanized monoclonal antibodies. More specifically, a “human” antibody, also called a “fully human” antibody, according to the invention, is an antibody that includes human framework regions and CDRs from a human immunoglobulin. For example, the framework and the CDRs of an antibody are from the same originating human heavy chain, or human light chain amino acid sequence, or both. Alternatively, the framework regions may originate from one human antibody, and be engineered to include CDRs from a different human antibody. “Humanizing substitutions” are amino acid substitutions in which the amino acid residue present at a particular position in the VH or VL domain of an antibody, such as an IL-38 antibody, is replaced with an amino acid residue which occurs at an equivalent position in a reference human VH or VL domain. The reference human VH or VL domain may be a VH or VL domain encoded by the human germline. Humanizing substitutions may be made in the framework regions and/or the CDRs of the antibodies, defined herein. A “humanized variant” is a variant antibody of the invention, which contains one or more “humanizing substitutions” relative to a reference antibody, wherein a portion of the reference antibody (e.g. the VH domain and/or the VL domain or parts thereof containing at least one CDR) has an amino acid derived from a non-human species, and the “humanizing substitutions” occur within the amino acid sequence derived from a non-human species

An antibody according to the invention may also be an “antigen-binding fragment”. An antigen-binding fragment refers to a polypeptide fragment of an immunoglobulin or antibody that binds antigen or competes with intact antibody (i.e., with the intact antibody from which they were derived) for antigen binding (i.e., specific binding to IL-38). As used herein, the term “fragment” of an antibody molecule includes antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means. Examples of immunoglobulin variants that are considered antibodies according to the invention include single-domain antibodies (such as VH domain antibodies), Fab fragments, Fab′ fragments, F(ab)′₂ fragments, single chain Fv proteins (“scFv”), and disulfide stabilized Fv proteins (“dsFv”). A VH single-domain antibody is an immunoglobulin fragment consisting of a heavy chain variable domain. An Fab fragment contains a monovalent antigen-binding immunoglobulin fragment, which can be produced by digestion of whole antibody with the enzyme papain to yield an intact light chain and a portion of one heavy chain. Similarly, an Fab′ fragment also contains a monovalent antigen-binding immunoglobulin fragment, which can be produced by digestion of whole antibody with the enzyme pepsin, followed by reduction, to yield an intact light chain and a portion of the heavy chain. Two Fab′ fragments are obtained per immunoglobulin molecule. An (Fab′)₂ fragment is a dimer of two Fab′ fragments, that can be obtained by treating whole antibody with the enzyme pepsin without subsequent reduction, so Fab′ monomers remain held together by two disulfide bonds. An Fv fragment is a genetically engineered fragment containing the variable region of the light chain and the variable region of the heavy chain expressed as two chains. A single chain (“sc”) antibody, such as scFv fragment, is a genetically engineered molecule containing the V_{L} region of a light chain, the V_{H} region of a heavy chain, linked by a suitable polypeptide linker as a genetically fused single chain molecule. A dimer of a single chain antibody, such as a scFV₂ antibody, is a dimer of a scFV, and may also be known as a “miniantibody”. A dsFvs variant also contains a V_{L} region of an immunoglobulin and a V_{H} region, but the chains have been mutated to introduce a disulfide bond to stabilize the association of the chains.

One of skill in the art will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules.

An antibody according to the invention may also comprise a “tagged” immunoglobulin CH3 domain to facilitate detection of the biologic against a background of endogenous antibodies. More particularly, a tagged CH3 domain is a heterogenous antibody epitope that has been incorporated into one or more of the AB, EF, or CD structural loops of a human IgG-derived CH3 domain. CH3 tags are preferably incorporated into the structural context of an IgG1 subclass antibody, other human IgG subclasses, including IgG2, IgG3, and IgG4, are also available according to the invention. Epitope-tagged CH3 domains, also referred to as “CH3 scaffolds” can be incorporated into any antibody of the invention having a heavy chain constant region, generally in the form of an immunoglobulin Fc portion. Examples of CH3 scaffold tags, and methods for incorporating them into antibodies are disclosed in International Patent Application No. PCT/US2019/032780. Antibodies used to detect epitope tagged CH3 scaffolds, and antibodies of the invention, that comprise epitope tagged CH3 scaffolds, are generally referred to herein as “detector antibodies”.

Therapeutic and diagnostic effectiveness of an antibody according to the invention correlates with its binding affinity for its target antigen. Binding affinity may be calculated by a modification of the Scatchard method described by Frankel et al., Mol. Immunol., 16:101-106, 1979. Alternatively, binding affinity may be measured by the dissociation rate of an antibody from its antigen. Various methods can be used to measure binding affinity, including, for example, surface plasmon resonance (SPR), competition radioimmunoassay, ELISA, and flow cytometry. An antibody that “specifically binds” an antigen is an antibody that binds the antigen with high affinity and does not significantly bind other unrelated antigens.

High affinity binding of an antibody to its antigen is mediated by the binding interaction of one or more of the antibody's CDRs to an epitope, also known as an antigenic determinant, of the antigen target. Epitopes are particular chemical groups or peptide sequences on a molecule that are antigenic, meaning they are capable of eliciting a specific immune response. An epitope that is specifically bound by an antibody according to the invention may be formed by a linear sequence of amino acids contained within IL-38. Such an epitope is called a “linear epitope”, and it may remain functional with respect to the specific binding of an antibody according to the invention to a denatured form of IL-38. Alternatively, the specific binding of an antibody according to the invention may depend on a particular three-dimensional structure of the IL-38 target, such that the contributing residues of an epitope are not necessarily in a linear sequence. In other words, an epitope of an antibody according to the invention may be a “conformational epitope”.

IL-38 may be present on the surface of various carcinoma cells, including carcinoma cells of epithelial origin or secreted into the extracellular environment by carcinoma cells, normal epithelial cells, or by cells of the immune system. Therefore, antibodies described herein can be included in compositions, which are useful for methods of diagnosing or treating various diseases where IL-38 acts to modulate disease progression. Those include, but are not limited to, cancer, including but not limited to cancers such as prostate, breast, renal, colorectal, pancreatic, melanoma, uterine, head & neck and lung cancer.

Therapeutic and diagnostic uses of antibodies according to the invention may include uses of immunoconjugates. As described herein, an immunoconjugate is a chimeric molecule, which comprises an effector molecule linked to an antibody according to the invention. As referred to herein, an effector molecule is the portion of an immunoconjugate that is intended to have a desired effect on a cell to which the immunoconjugate is targeted, or an effector molecule may serve to increase the half-life or bioavailability of an antibody according to the invention. General examples of effector molecules include therapeutic agents, (such as toxins and chemotherapeutic drugs), diagnostic agents, (such as detectable markers), and half-life and bioavailability-enhancing molecules, (such as lipids or polyethylene glycol).

Effector molecules can be conjugated to antibodies according to the invention using any number of means known to those of skill in the art, including covalent and noncovalent attachment means. The procedure for attaching an effector molecule to an antibody may vary according to the chemical structure of the effector. Polypeptides typically contain a variety of functional groups, such as a carboxylic acid (COOH) group, a free amine (—NH₂), and a sulfhydryl (SH) group, which are available for reaction with a suitable functional group on an antibody to result in the binding of the effector molecule. Alternatively, an antibody according to the invention can be derivatized to expose, or attach, additional reactive functional groups. Derivatization may involve attachment of any of a number of known linker molecules, which serve to join an antibody to an effector molecule.

A linker molecule is capable of forming covalent bonds to the antibody and effector molecule. Suitable linkers include, but are not limited to, straight or branched-chain carbon linkers, heterocyclic carbon linkers, or peptide linkers. If an effector molecule is a polypeptide, a linker may be joined to the constituent amino acids of the polypeptide through their side groups, such as through a disulfide linkage to cysteine, or to the alpha carbon amino and carboxyl groups of the terminal amino acids. Recombinant technology may be used to make two or more polypeptides, including linker peptides, into one contiguous polypeptide molecule.

An effector molecule may also be contained within a directly attached, or linked encapsulation system, that shields the effector molecule from direct exposure to the circulatory system. Means of preparing liposomes attached to antibodies are well known to those of skill in the art (see, for example, U.S. Pat. No. 4,957,735; and Connor et al., Pharm Ther 28:341-365, 1985).

The effector molecules of immunoconjugates according to the invention are generally useful for the treatment of cancer, and diseases characterized by abnormal cell growth, generally. Accordingly, the effector molecules of immunoconjugates according to the invention can be chemotherapeutic agents, including: small molecule drugs; nucleic acids, such as antisense nucleic acids, derivatized oligonucleotides for covalent cross-linking with single or duplex DNA, and triplex forming oligonucleotides; proteins; peptides; amino acids, and amino acid derivatives; glycoproteins; radioisotopes; lipids; carbohydrates; recombinant viruses; and toxins, such as, but not limited to, abrin, ricin, Pseudomonas exotoxin (“PE”, such as PE35, PE37, PE38, and PE40), diphtheria toxin (“DT”), botulinum toxin, saporin, restrictocin, gelonin, bouganin, and modified toxins thereof.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of a linker to release the effector molecule from an antibody according to the invention may be prompted by enzymatic activity or conditions to which the immunoconjugate is subjected either inside the target cell or in the vicinity of the target site. Alternatively, after specifically binding its target antigen, an antibody according to the invention can be internalized by the cell expressing the target antigen.

Therapeutic antibodies according to the invention, including therapeutic immunoconjugates, can be used in methods for preventing, treating, or ameliorating a disease in a subject. In certain embodiments of the invention, antibodies according to the invention can be used for preventing, treating, or ameliorating cancer in a subject. For example, antibodies according to the invention can be used to prevent, treat or ameliorate, cancer, including but not limited to, cancers of the prostate, breast, kidney, colon and rectum, pancreas, skin uterus, head & neck and lung.

“Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease. With respect to the use of antibodies according to the invention to prevent, treat or ameliorate cancer, signs or symptoms of the disease may correlate to tumor burden or the number or size of metastases.

A method for preventing, treating, or ameliorating cancer may require the administration of a composition, comprising an effective amount of an antibody according to the invention, to a subject to inhibit tumor growth or metastasis, comprising selecting a subject with a cancer characterized by tumor cells that expresses the antigen target of the antibody, or otherwise present cell membrane-associated target antigens of an antibody according to the invention. For example, an antibody according to the invention can contact a tumor cell via binding to its target antigen, to modulate, inhibit, or neutralize the target antigen's function. An antibody according to the invention may also deliver cytotoxic therapy upon binding to its target antigen on the surface of a tumor cell.

An antibody according to the invention can bind a target antigen in a fluid, such as, but not limited to, blood or a blood derivative, like plasma and serum, or a fluid within a tumor microenvironment. As is the case with a cell membrane-attached target antigen, binding of an antibody according to the invention to a secreted target antigen can result in the modulation, inhibition, or neutralization of the target antigen's biological function. Thus, the binding of an antibody according to the invention to its target antigen in solution, can, for example, modulate, inhibit, or neutralize its target antigen's activity, or the membrane-bound vesicle's activity, in vivo.

There are also uses of antibodies according to the invention, in which, an antibody binds a target antigen that is associated with an extracellular matrix (“ECM”) or ECM protein. For example, an antibody according to the invention may bind to a target antigen that is, itself associated with an ECM that migrating or differentiating endothelial cells are attached, or would be expected to encounter, to modulate, inhibit, or neutralize its target. The presence of ECM-associated target antigen may correlate with various disease states, including diseases associated with the presence of ECM-associated target antigen in the tumor microenvironment.

As stated above, antibodies disclosed herein can be administered to slow or inhibit the growth of primary tumors or inhibit the metastasis of various types of tumors. For example, antibodies according to the invention can be administered to slow or inhibit the growth or metastasis of cancers, including but not limited to, prostate, breast, renal, colorectal, pancreatic, melanoma, uterine, head & neck and lung cancer. In these applications, a therapeutically effective amount of an antibody is administered to a subject in an amount sufficient to inhibit growth, replication or metastasis of cancer cells, or to inhibit a sign or a symptom of the cancer. Suitable subjects may include those diagnosed with a cancer, in which the tumor cells express a target antigen of an antibody according to the invention. A therapeutically effective amount of an antibody according to the invention will depend upon the severity of the cancer, and the general state of the patient's health. A therapeutically effective amount of the antibody is that which provides either subjective relief of a symptom(s) or an objectively identifiable improvement as noted by a clinician or other qualified professional.

Antibodies according to the invention, that are administered to subjects in need thereof, are formulated into compositions. More particularly, the antibodies can be formulated for systemic administration, or local administration, such as intra-tumor administration. For example, an antibody according to the invention may be formulated for parenteral administration, such as intravenous administration. The compositions can be prepared in unit dosage forms for administration to a subject. The amount and timing of administration are at the discretion of the treating clinician to achieve the desired outcome.

Administration of antibodies according to the invention can also be accompanied by administration of other anti-cancer agents or therapeutic treatments, such as surgical resection of a tumor. Any suitable anti-cancer agent can be administered in combination with the antibodies disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, immune modulators, anti-hormones (e.g. anti-androgens) and anti-angiogenesis agents. Other anti-cancer treatments include radiation therapy and other antibodies that specifically target cancer cells.

The compositions for administration can include a solution of the antibody dissolved in a pharmaceutically acceptable carrier, such as an aqueous carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, or glycerol as a vehicle. For solid compositions, such as powder, pill, tablet, or capsule forms, conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. The foregoing carrier solutions are sterile and generally free of undesirable matter, and may be sterilized by conventional, well known sterilization techniques. The compositions may contain pharmaceutically acceptable auxiliary substances as required to approximate physiological conditions such as pH adjusting and buffering agents, and toxicity adjusting agents such as sodium acetate, sodium chloride, potassium chloride, calcium chloride, and sodium lactate. The concentration of antibody in these formulations can vary widely, and will be selected primarily based on fluid volumes, viscosities, body weight and the like in accordance with the particular mode of administration selected and the subject's needs.

Options for administering an antibody according to the invention include, but are not limited to, administration by slow infusion, or administration via an intravenous push or bolus. Other options for antibody administration may be optimized for intraocular administration. Prior to being administered, an antibody composition according to the invention may be provided in lyophilized form, and rehydrated in a sterile solution to a desired concentration before administration. The antibody solution may then be added to an infusion bag containing 0.9% sodium chloride, USP, and in some cases administered at a dosage of from 0.5 to 15 mg/kg of body weight. In one example of administration of an antibody composition according to the invention, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Antibody compositions according to the invention may also be controlled release formulations. Controlled release parenteral formulations, for example, can be made as implants, or oily injections. Particulate systems, including microspheres, microparticles, microcapsules, nanocapsules, nanospheres, and nanoparticles, may also be used to deliver antibody compositions according to the invention. Microcapsules, as referred to herein, contain an antibody according to the invention as a central core component. In microspheres, an antibody according to the invention is dispersed throughout the particle. Particles, microspheres, and microcapsules smaller than about 1 μm are generally referred to as nanoparticles, nanospheres, and nanocapsules, respectively.

As described above, antibodies according to the invention may also be useful for diagnosing or monitoring the presence of a pathologic condition, such as, but not limited to cancer of the prostate, breast, kidney, colon and rectum, pancreas, skin, uterus, head & neck and lung. More particularly, methods of the invention are useful for detecting expression of the antigen target of an antibody according to the invention. Detection may be in vitro or in vivo. Any tissue sample may be used for in vitro diagnostic detection, including, but not limited to, tissue from biopsies, autopsies and pathology specimens. Biological samples include sections of tissues, for example, frozen sections taken for histological purposes. Biological samples further include body fluids, such as blood, serum, plasma, sputum, spinal fluid or urine.

A method determines if a subject has a disease by contacting a sample from the subject with an antibody according to the invention; and detecting binding of the antibody to its target antigen present in the sample. An increase in binding of the antibody to its target antigen in the sample, as compared to binding of the antibody in a control sample identifies the subject as having a disease associated with IL-38 expression, such as, cancer or any other type of disease that expresses IL-38. In general, a control sample is a sample from a subject without disease.

Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (e.g., severity) of a pathologic condition, such as pancreatic cancer or metastasis.

Antibodies of the invention can be linked to a detectable label to form immunoconjugates that are useful as diagnostic agents. A detectable label, as referred to herein, is a compound or composition that is conjugated directly or indirectly to an antibody according to the invention, for the purpose of facilitating detection of a molecule that correlates to presence of a disease, such as, for example, a tumor cell antigen that is the antigen target of an antibody according to the invention. Detectable labels useful for such purposes are well known in the art, and include: radioactive isotopes, such as ³⁵S, ¹¹C, ¹³N, ¹⁵O, ¹⁸F, ¹⁹F, technetium-99m (^{″99m}Tc), ¹²⁴I, ¹³¹I, ⁸⁹Zr, ³H, ¹⁴C, ¹⁵N, ⁹⁰Y, ¹¹¹In and ¹²⁵I; fluorophores;

chemiluminescent agents; enzymatic labels, such as horseradish peroxidase, beta-galactosidase, luciferase, alkaline phosphatase; biotinyl groups; predetermined polypeptide epitopes recognized by a secondary reporter, such as a leucine zipper pair sequences, binding sites for secondary antibodies, metal binding domains, epitope tags; and magnetic agents, such as gadolinium chelates. A labeled antibody according to the invention may also be referred to as a “labeled antibody”, or more specifically a “radiolabeled antibody”. For some antibodies according to the invention, labels are attached by spacer arms of various lengths to reduce potential steric hindrance.

A diagnostic method comprising a step of using an antibody according to the invention may, in certain applications, be an immunoassay. While the details of immunoassays may vary with the particular format employed, a method of detecting the antigen target of an antibody according to the invention in a biological sample generally includes the steps of contacting the biological sample with the antibody which specifically reacts with the antigen, under immunologically reactive conditions to form an immune complex. The presence of the resulting immune complex can be detected directly or indirectly. In other words, an antibody according to the invention can function as a primary antibody (1° Ab) in a diagnostic method, and a labeled antibody, specific for the antibody according to the invention, functions as the 2° Ab. In the case of indirect detection of an immune complex, the use of an antibody according to the invention for a diagnostic method will also include the use of a labelled secondary antibody (2° Ab) to detect binding of the primary antibody—the antibody according to the invention—to its target antigen. Suitable detectable labels for a secondary antibody include the labels, described above, for directly labeled antibodies according to the invention. A 2° Ab, used in a diagnostic method according to the invention, may also be a “detector antibody”, as defined, above, for use in conjunction with an antibody according to the invention that contains a CH3 epitope tag, as described in International Patent Application No. PCT/US2019/032780.

Antibodies according to the invention can also be used for fluorescence activated cell sorting (FACS). A FACS analysis of a cell population employs a plurality of color channels, low angle and obtuse light-scattering detection channels, and impedance channels, among other more sophisticated levels of detection, to separate or sort cells (see U.S. Pat. No. 5,061,620).

Reagents used in a diagnostic application of an antibody according to the invention, as described above, may be provided in a kit for detecting the antigen target of an antibody according to the invention a biological sample, such as a blood sample or tissue sample. Such a kit can be used to confirm a cancer diagnosis in a subject. For example, a diagnostic kit comprising an antibody according to the invention can be used to perform a histological examination for tumor cells in a tissue sample obtained from a biopsy. In a more particular example, a kit may include antibodies according to the invention that can be used to detect lung cancer cells in tissue or cells obtained by performing a lung biopsy. In an alternative, particular example, a kit may include antibodies according to the invention that can be used to detect pancreatic cancer cells in a tissue biopsy. Kits for detecting an antigen target of an antibody according to the invention will typically comprise an antibody according to the invention in the form of a monoclonal antibody, or an antigen-binding fragment thereof, such as an scFv fragment, a VH domain, or a Fab. The antibody may be unlabeled of labeled by a detectable marker, such as a fluorescent, radioactive, or an enzymatic label, as described above. A kit also generally includes instructional materials disclosing means of use of an antibody according to the invention. The instructional materials may be written, in an electronic form, such as a portable hard drive, and the materials also be visual, such as video files. Instructional materials may also refer to a website or link to an application software program, such as a mobile device or computer “app”, that provides instructions. A kit may also include additional components to facilitate the particular application for which the kit is designed. For example, a kit may also contain a means of detecting a label (such as enzyme substrates for enzymatic labels, filter sets to detect fluorescent labels, appropriate secondary labels such as a secondary antibody, or the like). Buffers and other reagents, which are routinely in methods of using an antibody according to the invention for diagnostic purposes.

Antibodies according to the invention can be produced by various recombinant expression systems. In other words, the antibodies can be produced by the expression of nucleic acid sequences encoding their amino acid sequences in living cells in culture. An “isolated” antibody according to the invention is one which has been substantially separated or purified away from other biological components environment, such as a cell, proteins and organelles. For example, an antibody may be isolated if it is purified to: i) greater than 95%, 96%, 97%, 98%, or 99% by weight of protein as determined by the Lowry method, and alternatively, more than 99% by weight; ii) a degree sufficient to obtain at least 15 residues of N-terminal or internal amino acid sequence by use of a spinning cup sequenator; iii) homogeneity by SDS-PAGE, under reducing or nonreducing conditions, using Coomassie blue or silver stain. Isolated antibody may also be an antibody according to the invention that is in situ within recombinant cells, since at least one component of the antibody's natural environment will not be present. Ordinarily, however, isolated antibody will be prepared by at least one purification step.

A variety of host-expression vector systems may be utilized to express an antibody according to the invention, by transforming or transfecting the cells with an appropriate nucleotide coding sequences for an antibody according to the invention. Examples of host-expression cells include, but are not limited to: Bacteria, such as E. coli and B. subtilis, which may be transfected with antibody coding sequences contained within recombinant bacteriophage DNA, plasmid DNA, or cosmid DNA expression vectors; Yeast, such as Saccharomyces and Pichia, transformed with recombinant yeast expression vectors containing antibody coding sequences; Insect cell systems, infected with recombinant virus expression vectors, such as baculovirus, containing antibody coding sequences; Plant cell systems infected with recombinant virus expression vectors, such as cauliflower mosaic virus (“CaMV”), or tobacco mosaic virus (“TMV”), containing antibody coding sequences; and Mammalian cell systems, such as, but not limited to COS, Chinese hamster ovary (“CHO”) cells, ExpiCHO, baby hamster kidney (“BHK”) cells, HEK293, Expi293, 3T3, NSO cells, harboring recombinant expression constructs containing promoters derived from the genome of mammalian cell, such as the metallothionein promoter or elongation factor I alpha promoter, or from mammalian viruses, such as the adenovirus late promoter, and the vaccinia virus 7.5K promoter. For example, mammalian cells such as Human Embryonic Kidney 293 (HEK293) or a derivative thereof, such as Expi293, in conjunction with a dual promoter vector that incorporates mouse and rat elongation factor 1 alpha promoters to express the heavy and light chain fragments, respectively, is an effective expression system for antibodies according to the invention, which can be advantageously selected, depending upon the use intended for the antibody molecule being expressed. Alternatively, a two-vector system that expresses the heavy and light chain fragments from separate plasmids under control of the cytomegalovirus (CMV) enhancer and promoter sequences, is an effective expression system for the antibodies, in conjunction with CHO cells, HEK cells, or their derivatives

When a large quantity of an antibody according to the invention is to be produced for the generation of a pharmaceutical composition of the antibody, vectors which direct the expression of high levels of readily purified fusion protein products may be desirable. Such vectors include, but are not limited to: a pUR278 vector (Ruther et al. EMBO J. 2:1791 (1983)), in which the antibody coding sequence may be ligated individually into the vector in frame with a lac Z coding region so that a fusion protein is produced; a pIN vector (Inouye & Inouye, Nucleic Acids Res. 13:3101-3109 (1985)

A host expression cell system may also be chosen which modulates the expression of inserted sequence(s) coding for an antibody according to the invention, or modifies and processes the gene product as desired. For example, modifications, including the glycosylation and processing, such as cleavage of protein products, may be important for the function of the protein. Indeed, different host cells have characteristic and specific mechanisms for the posttranslational processing and modification of proteins and gene products. To this end, eukaryotic host cells, which possess appropriate cellular machinery for proper processing of a primary transcript, as well as the glycosylation and phosphorylation of a gene product according to the invention may be used.

The vector used to produce an antibody according to the invention comprises a nucleic acid molecule encoding at least a portion of that particular antibody. For example, such a nucleic acid sequence can comprise a DNA sequence corresponding to any polynucleotide sequences comprising VH and VL domains therein, including codon optimized sequences, or a portion thereof. Thus, a first nucleic acid encoding at least a portion of an antibody according to the invention, that is operably linked with a second nucleic acid sequence that is placed in a functional relationship with the first nucleic acid sequence, such as a promoter, is a nucleic acid according to the invention. An operable linkage exists if a linked promoter sequence affects the transcription or expression of the coding sequence. Generally, operably linked DNA sequences are contiguous and, may also join two, or more, protein-coding regions, in the same reading frame.

When a nucleic acid comprising a DNA sequence according to the invention is substantially separated or purified away from other biological components in the environment, such as a cell, other chromosomal and extra-chromosomal DNA and RNA, proteins and organelles, it may be considered to be an “isolated nucleic acid” according to the invention. For example, a nucleic acid, which has been purified by standard purification methods, is an isolated nucleic acid.

Nucleic acids according to the invention also include degenerate variants of nucleotides encoding an antibody according to the invention. More particularly, a “degenerate variant” refers to a polynucleotide, which encodes an antibody according to the invention, but is degenerate as a result of the genetic code. All degenerate nucleotide sequences are included, according to the invention, as long as the amino acid sequence of the encoded antibody specifically binds the antigen target of an antibody according to the invention.

### EXAMPLES

The following Examples describe the isolation and characterization of IMM20130, an antibody which binds to an epitope on IL-38; an evaluation of the immunosuppressive effects of IL-38; the generation of additional anti-IL-38 antibodies; and their characterization both in vitro and in vivo. IL-38, can, in certain contexts, be soluble, or associated with a cell membrane, including at a cell surface, including in context of a multi-protein complex.

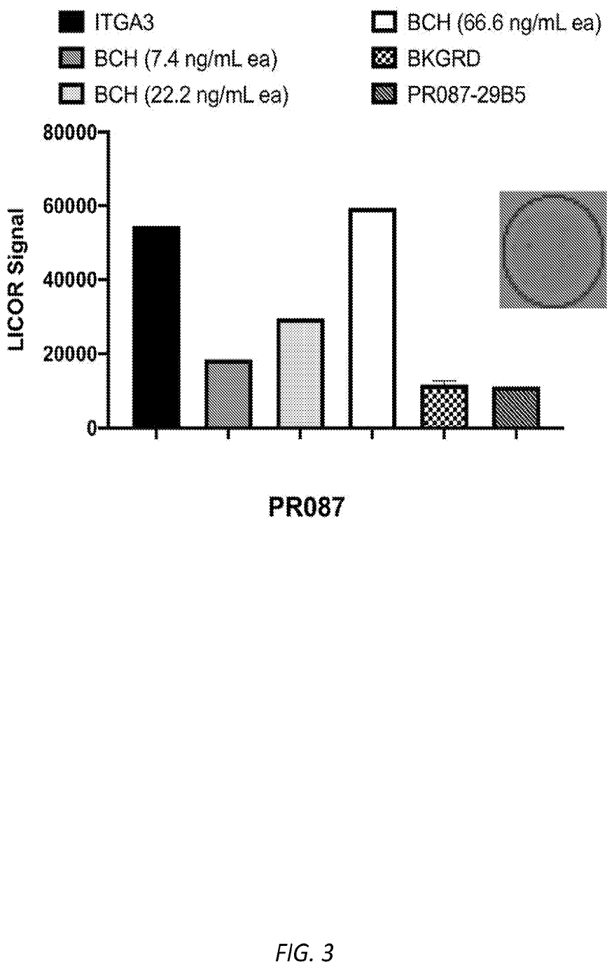

Example 1. Isolation of a human hybridoma producing an antibody that binds the surface of intact human cancer cells. PR087-29B5 hybridoma cells were generated from the fusion of human B cells isolated from the lymph node of a head and neck cancer patient, with the B56T fusion partner. Fusion of human B cells with B56T was carried out by electrofusion essentially as described in USPTO #EP2242836 “Method of making hybrid cells that express useful antibodies”. Post-fusion, hybridomas were plated and allowed to grow for approximately two weeks. Conditioned medias from IgG/A-positive hybridomas were then collected and screened for the ability of the antibodies to bind to the surface of cancer cell lines. Binding of PR087-29B5-produced Abs to pools of live, intact cancer cell lines was detected using fluorophore-labelled anti-human IgG secondary Abs and a LI-COR Odyssey™ Sa imaging system configured for 96-well plates. Prior to screening, the cancer cells were mixed in equal proportions and the pools were aliquoted into 96-well plates and allowed to attach for 24 hours. Hybridoma supernatants were incubated with cells and binding to the cancer cell lines was assessed relative to a positive control comprising a mixture of anti-basigin, anti-EGFR, and anti-ERBB2 (BCH) antibodies in equal ratios. The BCH positive control was incubated with cells at 66.6, 22.2, and 7.4 ng/mL of each antibody. An anti-Integrin (ITGA3) antibody (20 ng/mL) was also used as a positive control. Secondary antibody alone was used as a negative control. The combination of controls provides a range of absolute signal intensities across both the cell line pools and the detection range of the LI-COR instrument. The BCH (7.4 ng/mL) positive control exhibited a signal approximately 160% that of the background, where the background was defined as the average of the signal found in the four wells of secondary alone controls. The signals across those four wells had a standard deviation of 8.5%. PR087-29B5 did not exhibit a signal above the level of background, but rather presented as a low level punctate staining pattern that was selected for subsequent follow-up (FIG. 3).

Example 2. The PR087-29B5 hybridoma produces an IgG comprising IGHV1/IGLV2 variable domains. Nucleotide sequences encoding the variable heavy chain (V_{H}) and variable light chain (V_{L}) domains of PR087-29B5 were obtained by RT-PCR amplification of RNA isolated from cells of the PR087-29B5 hybridoma line and subjecting the resulting antibody cDNA to sequencing reactions. SEQ ID NO: 1 corresponds to the nucleotide sequence of the V_{H} and SEQ ID NO: 3 corresponds to the nucleotide sequence of the V_{L} of PR087-29B5 isolated from the hybridoma. SEQ ID NO: 2 and SEQ ID NO: 4 correspond to the corresponding amino acid sequences of the V_{H} and V_{L} of PR087-29B5 isolated from the hybridoma. IGHV1-18 and IGKV3-20 gene assignments were predicted based upon homology to known germline gene sequences and were used to generate the 5′ ends of the V_{H} and V_{L} to create full-length coding sequences represented by SEQ ID NO: 5 and SEQ ID NO: 8, which encode the amino acid sequences SEQ ID NO: 7 and SEQ ID NO: 10, respectively. A two plasmid system was used to facilitate recombinant expression of antibodies that contain the variable domains of PR087-29B5 with IgG1 heavy chain and kappa light chain constant domains. Codon optimization was carried out on SEQ ID NO: 5 and a nucleotide fragment corresponding to SEQ ID NO: 6, which encodes the amino acid sequence corresponding to SEQ ID NO: 7, was synthesized to facilitate expression of antibodies comprising the VH domain of PR087-29B5. Codon optimization was also carried out on SEQ ID NO: 8 and a nucleotide fragment corresponding to SEQ ID NO: 9, which encodes SEQ ID NO: 10, was synthesized to facilitate expression of antibodies comprising the V_{L} domain of PR087-29B5. Vectors expressing either the heavy chain or light chain of PR087-29B5 were generated by synthesis and cloning of V_{H} and V_{L} domains into a two vector system that encode for a full-length IgG1 antibody comprised of amino acid sequences corresponding to SEQ ID NO: 12 and SEQ ID NO: 14. Antibodies containing the PR087-29B5 V_{H} and V_{L} domains, were expressed recombinantly by transient transfection into mammalian cell lines, such as Chinese Hampster Ovary (CHO) and human embryonic kidney (HEK), using standard conditions. Recombinant antibodies, referred to as IMM20130, were purified from conditioned media by affinity chromatography using techniques well known to one of ordinary skill in the art.

Example 3. IMM20130 Ab binds an epitope on IL-38. To identify the target antigen bound by IMM20130, the antibody was screened against CDI HuProt arrays where target proteins were spotted in their native conformation. More specifically, IMM20130 was incubated (1 microgram/mL) overnight at 4° C. against the native CDI HuProt arrays. Slides were washed and IMM20130 binding was detected with an Alexa-647 conjugated anti-H+L secondary antibody. Non-specific hits that were bound by the secondary antibody were eliminated from any analysis. Selective binding to target proteins was analyzed by a combination of Z-score, to determine reproducibility of binding to replicates on each slide, and S-score, to determine difference in selectivity versus possible targets. An S-score>3 between the top and second-ranked hits is considered as highly specific for the top hit.

IMM20130 bound selectively to IL1F10/IL-38 found on the native array. IL-38 represented the top hit on the CDI array with a Z-score of 119.635 and an S-score of 51.643. See Table 1.

TABLE 1
IMM20130 binding to human proteome in protein microarray format
CDI Native Array

| Rank | Protein | Z Score | S Score | F635 |
|---|---|---|---|---|
| 1 | IL1F10/IL38 | 119.635 | 51.643 | 18395.5 |
| 2 | FERD3L | 67.992 | 32.891 | 10473.5 |
| 3 | STIP1 | 35.101 | 8.615 | 5428 |
| 4 | DNAJC7 | 26.486 | 6.004 | 4106.5 |
| 5 | STIP1 | 20.482 | 8.866 | 3185.5 |
| 5 | TTC1 | 11.616 | 0.251 | 1825.5 |
| 7 | ZNF41 | 11.365 | 1.829 | 1787 |
| 8 | PPIA | 9.536 | 0.029 | 1506.5 |
| 9 | HIST1H1B | 9.507 | 0.049 | 1502 |
| 10 | FAM104B | 9.458 | 0.094 | 1494.5 |

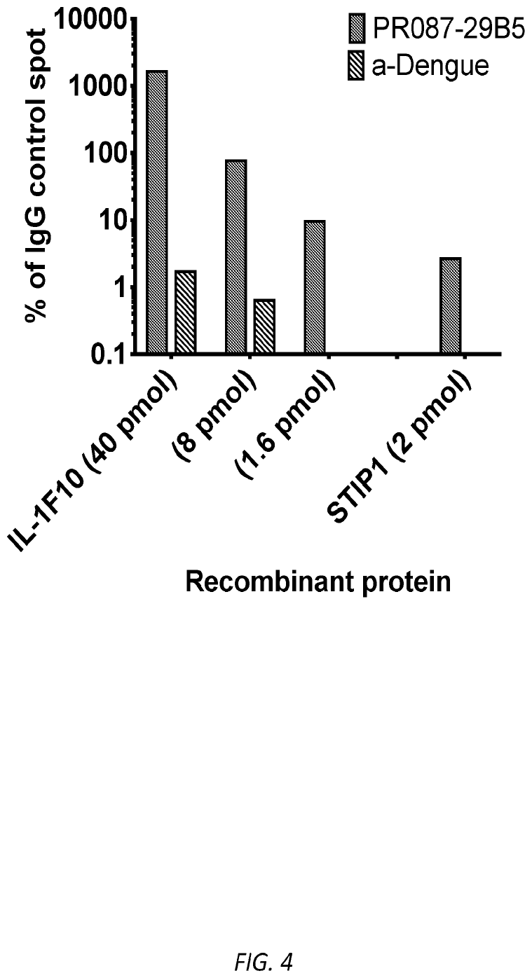

Binding of IMM20130 to recombinant IL-38 was confirmed by dot-blot analysis. Increasing doses of recombinant human IL-38 (NovusBio, catalog number NBP2-22645) was spotted onto nitrocellulose and incubated with IMM20130. As shown in FIG. 4, IMM20130 interacted with IL-38 in a dose-dependent manner. A commercial anti-IL-38 antibody served as the positive control for the assay and an anti-Dengue antibody of the same isotype served as a negative control. The recombinant protein STIP1, a lower level hit in the original CDI array, was used as a non-specific control.

Binding of IMM20130 to IL-38 (NovusBio, catalog number NBP2-22645) was quantified by surface plasmon resonance (SPR). Briefly, IMM20130 was diluted in SPR running buffer (10 mM HEPES, pH7.4, 150 mM NaCl, 0.0005% Tween-20, 0.2% bovine serum albumin) to final concentrations of 150 nM and 25 nM and captured at four different surface densities on an anti-human Fc coated CM5 sensor chip. Surface densities ranged from 600 to 3200 RU. IL-38 (NovusBio, catalog number NBP2-22645) was diluted in SPR running buffer to a concentration of 600 nM and a 3-fold dilution series was run across the four different IMM20130 density surfaces. Data were collected at 25° C. Data from all four surfaces were fitted to a 1:1 interaction model yielding the rate constants depicted in Table 2.

TABLE 2
SPR Quantification of IMM20130 binding to human IL-38
SPR analysis

| kₐ | k_{d} | K_{D} |
|---|---|---|
| 4.98(8)e3 | 6.80(1)e−5 | 1.36(2)e−8 |

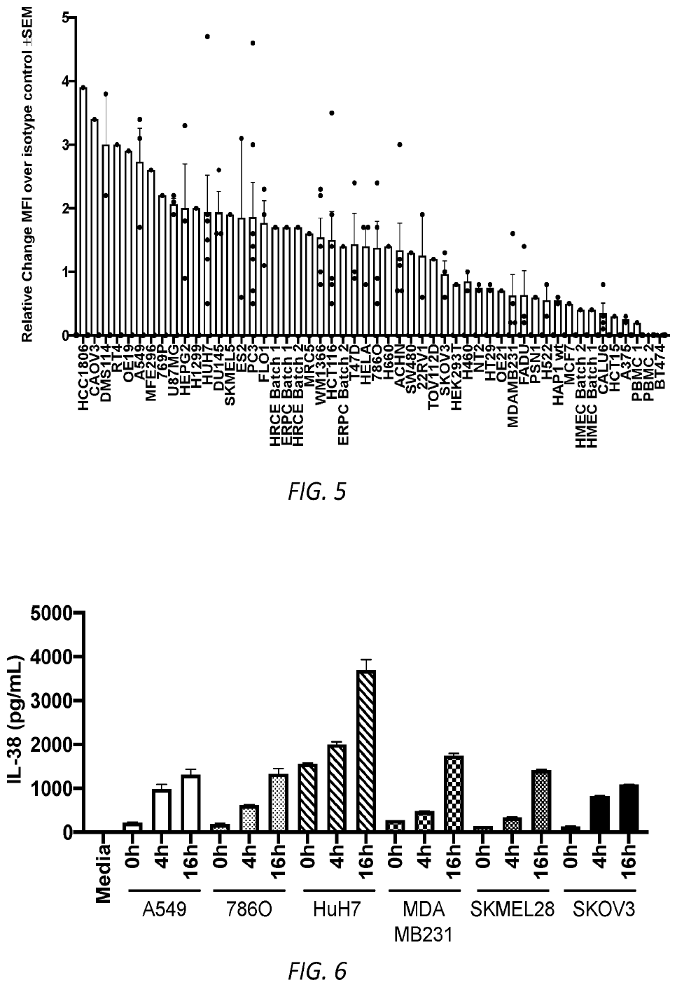

IMM20130 also specifically bound to the surface of several endogenously-expressing cell lines (FIG. 5). Live cells were stained with IMM20130 or isotype control and a fluorochrome-conjugated anti-human secondary antibody. Propidium iodide was used to exclude dead cells. Data is expressed as fold change of MFI over isotype control. Since IL-38 can be secreted under apoptotic conditions (Mora et al, 2016), several IMM20130-binding cancer cell lines were tested for their ability to secrete IL-38. Cancer cell lines were treated with 20 ng/mL TNFα and 10 μg/mL cycloheximide for the indicated time period. For 0 h and 4 h timepoints, cells were cultured in plain RPMI for 16 hours post-treatment. For the 16 h timepoint, cells were cultured with TNFa and cycloheximide for the full 16 hours in plain RPMI. The concentration of IL-38 in the supernatants was determined by direct ELISA using a protocol adapted from Mora et al 2016. In brief, 100 μL of supernatant was added to a high binding 96 well plate (Corning) and compared to a standard curve of seven two-fold dilutions of recombinant IL-38 (Adipogen, catalog number AG-40A-0191-C050) in plain RPMI. Plates were incubated overnight at 4° C. Wells were blocked with PBS 2% BSA, washed 3 times with PBS 0.05% Tween, and incubated with a rat anti-human IL-38 antibody (R&D Systems) for 2 hours at room temperature. After 3 washes, wells were incubated with a biotinylated anti-rat secondary antibody (Invitrogen) for 2 hours at room temperature. Following 3 washes, streptavidin-HRP (R&D Systems) in PBS 2% BSA was added for 20 minutes. After 3 washes, 100 μL of OPD substrate diluted in phospho-citrate/sodium perborate buffer was added per well, developed for 5-30 minutes, and absorbance was measure at 450 nm. Indeed, multiple cancer cell lines secreted IL-38 under apoptosis-inducing conditions (FIG. 6), identifying tumor cells as a potential source of IL-38.

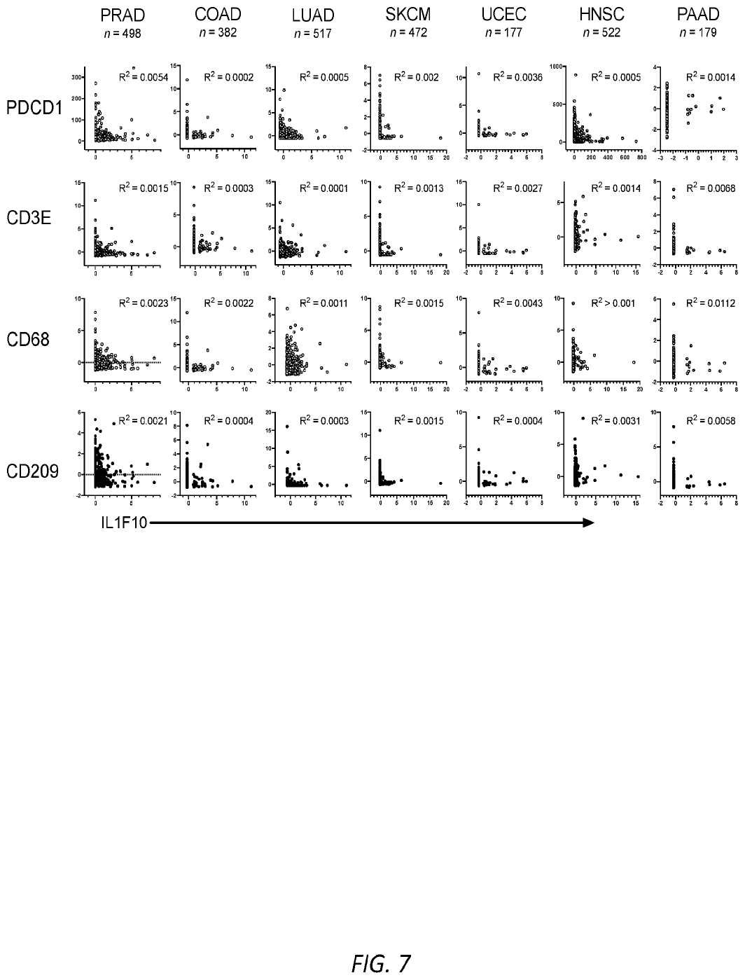

Example 4. IL-38 is a pro-tumorigenic, immunosuppressive cytokine that inhibits inflammatory responses. After assessing the expression of IL-38 in various cancer cell lines with IMM20130, the TCGA database was used to assess the effect of IL-38 on the tumor microenvironment. Gene expression analysis was performed using TCGA Firehouse Legacy datasets from the indications specified above. The number of samples in each dataset is indicated along with the R-squared values for each analysis. RNA_Seq_v2_mRNA_median_Zscore data was used for data analysis. In multiple cancer types, expression of IL-38 correlated with reduced expression of genes associated with immune cell types essential for an effective anti-tumor response, including T cells and myeloid cells (FIG. 7), suggesting that IL-38 can play an important role in suppressing immune cell infiltration into the tumor microenvironment.

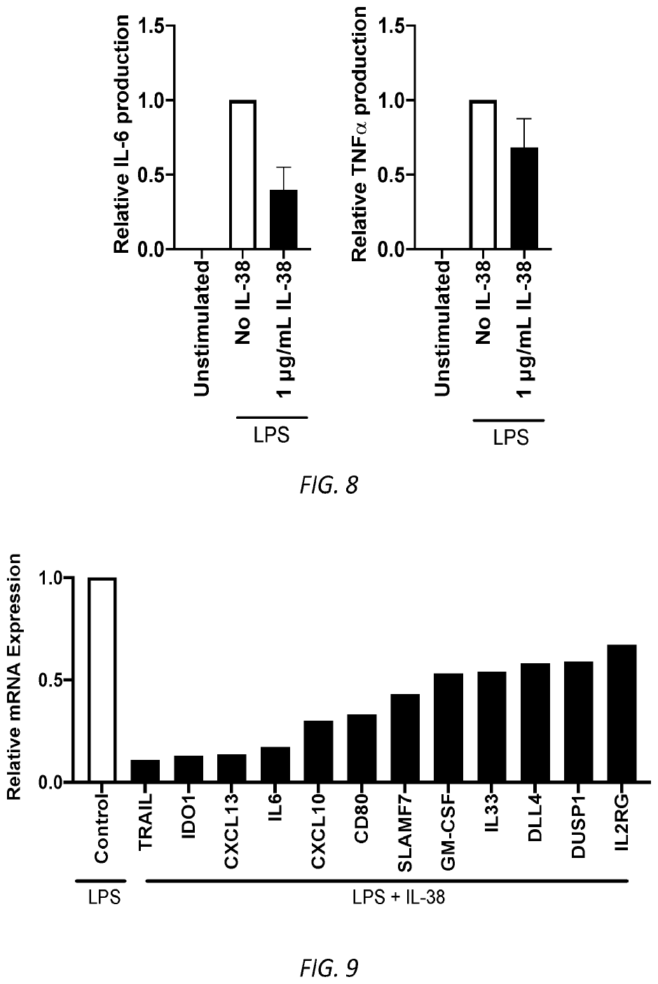

To identify how IL-38 could suppress the immune system, an in vitro model was established using the THP-1 monocyte cell line (ATCC, catalog number TIB-202). THP-1 monocytes were differentiated to macrophages by culturing with 100 nM PMA for 72 hours. After differentiation, PMA was removed, macrophages were washed with PBS, and cultured in plain RPMI with or without 1 μg/mL recombinant full length human IL-38 (Adipogen, catalog number AG-40A-0191-C050) for 24 hours. To stimulate macrophages and induce production of inflammatory cytokines, 10 ng/mL LPS was added for an additional 24 hours. Supernatants were harvested and cytokine expression was measured using CBA Human Inflammatory Cytokines Kit (BD Biosciences, catalog number 551811) per manufacturer's instructions. Treatment of THP-1 macrophages with IL-38 resulted in a reduction in several inflammatory cytokines, namely IL-6 and TNFα (FIG. 8). In order to have a more complete understanding of the effect of IL-38 on macrophage inflammatory responses, THP-1 cells was assayed for RNA expression of important inflammatory markers using the Nanostring PanCancer IO 360 Gene Expression Panel. THP-1 cells were differentiated and stimulated as described for FIG. 8. After LPS stimulation, cells were harvested and RNA was isolated using RNeasy kit (Qiagen). Nanostrings PanCancer IO 360 Gene Expression Panel was used to assess gene expression using the nCounter Platform (Nanostring Technologies). nSolver software was used for data analysis. The LPS-stimulated sample was used to normalize gene expression to 1. Several important inflammatory markers were reduced in IL-38-treated cells, including pro-inflammatory M1 macrophage markers (CD80, IL-6) and chemokines important for immune cell recruitment (CXCL10, CXCL13) (FIG. 9).

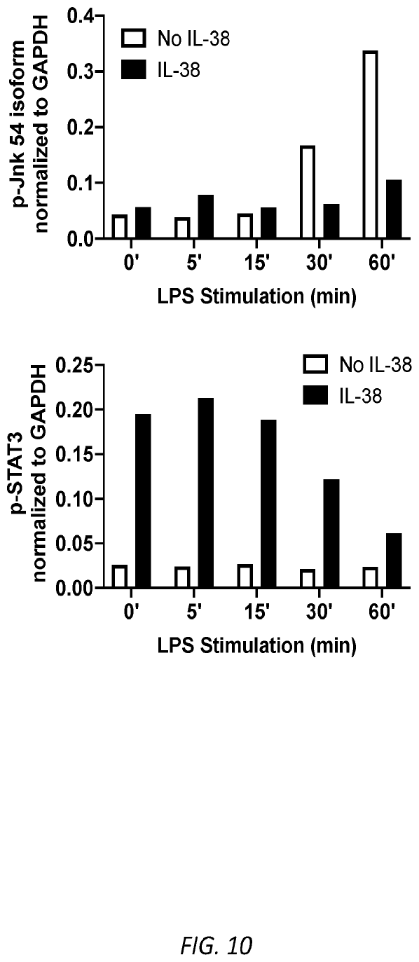

To determine how IL-38 suppresses inflammatory responses in THP-1 cells, phosphorylation of key signaling proteins was measured. Using the in vitro system described in FIG. 8, differentiated THP-1 macrophages pretreated with IL-38 were stimulated with 10 ng/mL LPS for various timepoints. Following stimulation, cells were lysed in 1% Triton-containing lysis buffer (Cell Signaling) with phosphatase and protease inhibitors. 20 μg per lane was loaded onto a 4% to 12% polyacrylamide gel (Invitrogen) and transferred onto a nitrocellulose membrane. The membrane was probed overnight with rabbit anti-human antibodies recognizing p-STAT3 and GAPDH and a mouse anti-human p-Jnk antibody (Cell Signaling). Then, the membranes were incubated with fluorescent anti-rabbit and anti-mouse secondary antibodies (LI-COR Biosciences) for 1 hour. Blots were scanned using LI-COR imaging system and quantification performed in Image Studio software (LI-COR Biosciences). Phosphorylation of Jnk was impaired in IL-38-treated THP-1 macrophages (FIG. 10). Conversely, phosphorylation of STAT3 was increased in IL-38-treated THP-1 macrophages prior to stimulation with LPS.

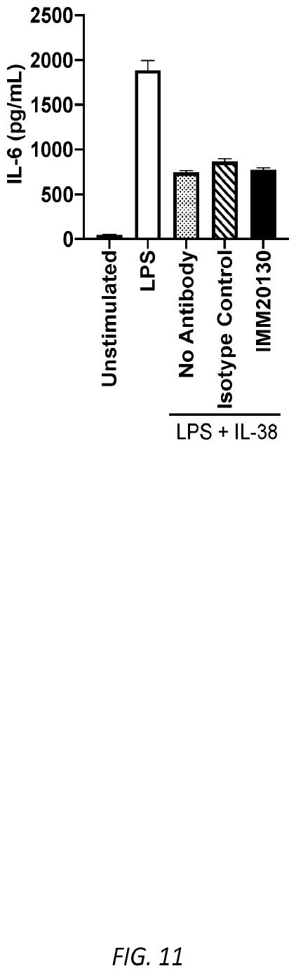

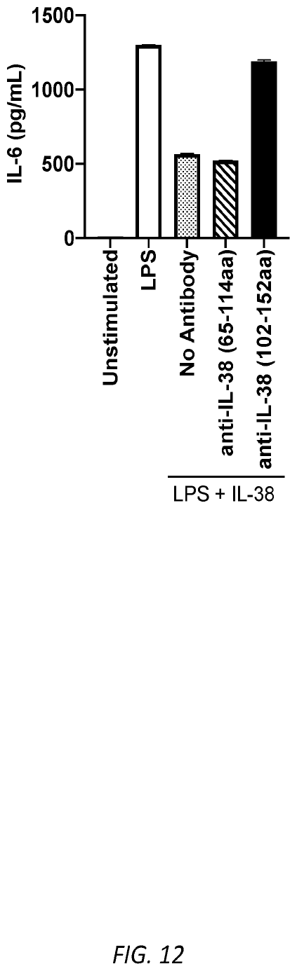

Example 5. Generation of anti-IL-38 antibodies that block IL-38 function. The in vitro system established in FIG. 8 was used to test the ability of IMM20130 to block the function of IL-38. THP-1 monocytes were differentiated into macrophages with 100 nM PMA for 72 hours. After differentiation, 1 μg/mL IL-38 (Adipogen, catalog number AG-40A-0191-C050) and 10 μg/mL of the indicated antibody were incubated at room temperature for 1 hour in plain RPMI. Macrophages were washed with PBS and cultured with the indicated IL-38/antibody-containing media for 24 hours. Subsequently, cells were stimulated with 10 ng/mL LPS for 24 hours, supernatants were harvested and IL-6 production was measured using the Human IL-6 DuoSet ELISA kit (R&D Systems). Inhibition of IL-38 should result in the restoration of IL-6 production in IL-38-treated, LPS-stimulated THP-1 cells to levels comparable to LPS-stimulated cells. However, IMM20130 was unable to restore IL-6 production of these cells (FIG. 11). As controls, two polyclonal antibodies raised against different portions of the IL-38 protein (Lifespan Biosciences, catalog numbers LS-C135753 and LS-C201139) were also tested for their ability to restore IL-6 production in this system. One polyclonal antibody raised against a portion of the C-terminus of IL-38 successfully restored IL-6 production of IL-38-treated, LPS-stimulated THP-1 macrophages (FIG. 12), indicating that IMM20130 binds to an epitope of IL-38 that does not block IL-38 function.

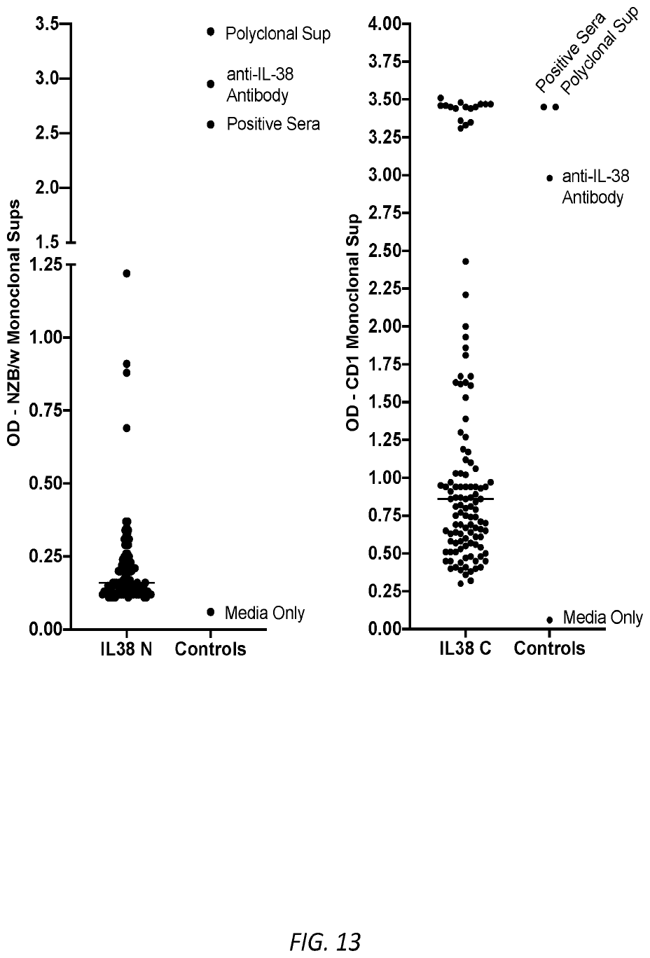

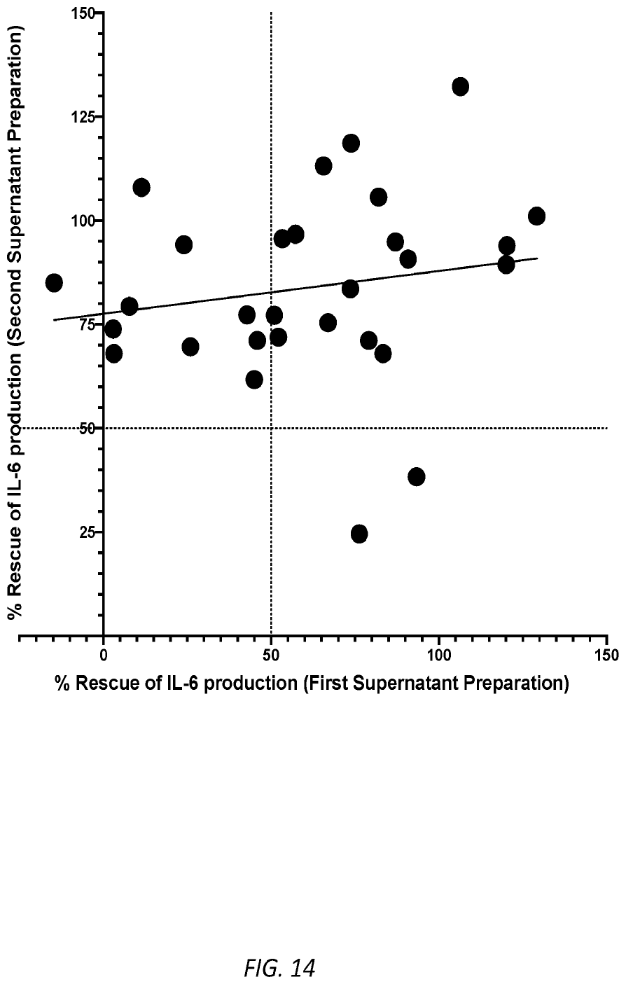

Although IMM20130 did not block the function of IL-38, it did identify IL-38 as an important modulator of the inflammatory response and a promising cancer target. Therefore, an antibody generation campaign was initiated to isolate anti-IL-38 antibodies that also block IL-38 function. NZB/w and CD-1 mice were immunized with full length recombinant IL-38. At Day 21 post immunization, mouse sera were assayed for the presence of anti-IL-38 antibodies by the Direct ELISA described in Example 2 using an HRP-conjugated anti-mouse secondary antibody (Jackson ImmunoResearch Laboratories, catalog number 115-035-071). Spleens from animals with the highest anti-IL-38 sera titer were fused with a myeloma cell line to generate a polyclonal hybridoma library. After polyclonal supernatants were confirmed to have anti-IL-38 antibodies by ELISA, individual hybridomas were single cell sorted and cultured in 96 well plates to generate antibody-containing monoclonal supernatants. A direct IL-38 ELISA was performed with the indicated controls on monoclonal supernatants from NZB/w and CD-1 mice-derived hybridomas as described in FIG. 6. Multiple monoclonal supernatants contained anti-IL-38 antibodies (FIG. 13). The selected IL-38 binding monoclonal supernatants were also tested for the ability to block IL-38 function in the in vitro system described in FIG. 11. IL-6 production was measured using two preparations of each monoclonal supernatant. Blocking efficiency was determined by normalizing IL-6 production of LPS-stimulated THP-1 cells to 100% and IL-6 production of IL-38-treated, LPS-stimulated THP-1 cells to 0%. Therefore, the percent rescue of each monoclonal supernatant was calculated as the IL-6 production of [(sample containing monoclonal sup, LPS, and IL-38)−(LPS, IL-38 Control)]/[(LPS alone control)−(LPS, IL-38 control)]. High blocking efficiency was observed with multiple monoclonal supernatants (FIG. 14) and these were selected for further development.

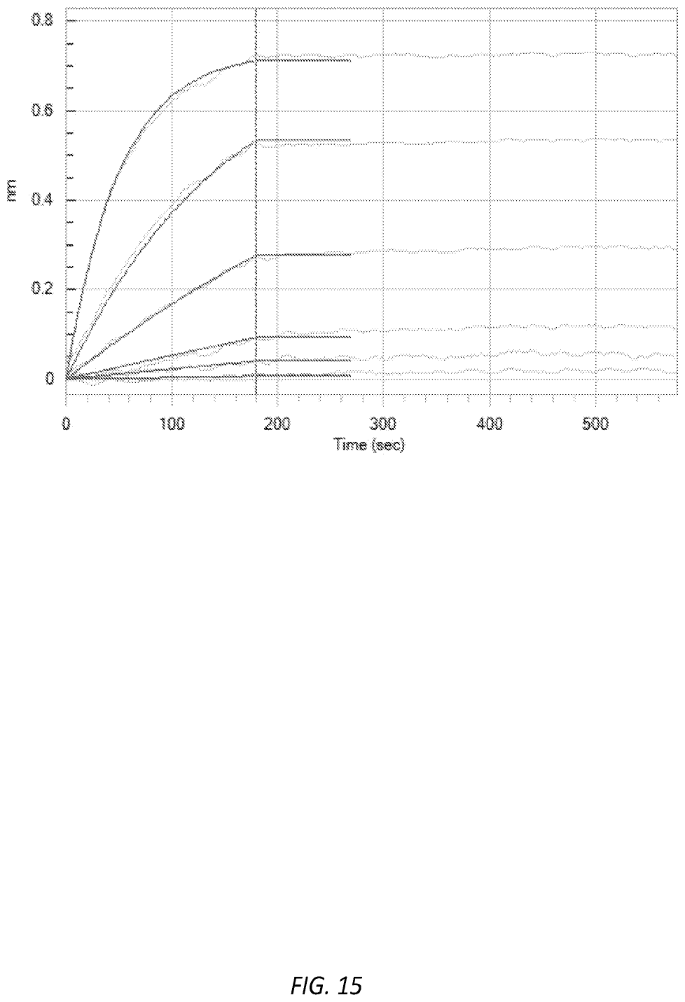

Antibody-containing solutions, including mouse hybridoma supernatants and purified antibodies, were tested on Octet Qke instrument using ForteBio's anti-mouse Fc (AMC) biosensors and soluble recombinant human (Adipogen, catalog number 40A-0191-C050) or mouse (Lifespan Biosciences, catalog number LS-G3934) IL-38 proteins that were serially diluted in the ForteBio's kinetics buffer. Antibody was loaded to AMC probes, blocked in the kinetics buffer for 1 min (baseline) and dipped into an appropriate IL-38 solution. IL-38 association to antibodies of interest was measured for 180 seconds at 28° C. Biosensors were subsequently dipped into kinetics buffer-containing wells and the protein dissociation was measured for 600 s. Raw traces were analyzed as shown in Table 3. KD, Kon and Kdis values were identified using the 1:1 built-in model of the ForteBio's Data Analysis 9 software. Binding kinetics of a selected anti-human IL-38 antibody are demonstrated in FIG. 15.

TABLE 3
Binding characteristics of lead candidate anti-IL-38 antibodies.

| | human IL-38 | | | mouse IL-38 | | |
|---|---|---|---|---|---|---|
| Antibody | Kon | Koff | KD | Kon | Koff | KD |

| | | | | |
|---|---|---|---|---|
| CD1-M3 | 2.64E+04 | 1.85E−05 | 7.21E−10 | 9.25E+03 |
| CD1-M8 | 2.80E+04 | | | |
| CD1-M26 | 3.50E+04 | 2.31E−04 | 6.60E−09 | |
| CD1-M27 | 1.66E+04 | | | |

To confirm specificity to IL-38, several lead candidate antibodies (CD1-M3, CD1-M8, NZB-M8) were analyzed in a High-Spec Cross-reactivity Assay using native HuProt arrays (CDI Laboratories). 1/mL of antibody was incubated overnight in a cold room, washed, and probed with anti-human secondary antibodies. Fluorescence intensity (F635) as an indicator of binding was measured for each spot. CDI software quantified the specificity of the antibody to each spot based on a Z-score. Z-score is defined as [F635−(Average F635 on array)]/(Standard deviation of F635 on array). S-score is defined as the difference between the Z-score of a given protein and the Z-score of the next highest protein. For CD1-M3 and CD1-M8, bound selectively to IL-38 on the native array with Z-scores of 139.533 and 142.421, respectively (Table 4).

TABLE 4
Binding of lead candidate antibodies to human
proteome in protein microarray format.

| | | | | |
|---|---|---|---|---|
| Rank | Protein | Z-score | s-score | F635 |

CD1-M3

| | | | | |
|---|---|---|---|---|
| 1 | IL1F10/IL38 | 139.533 | 90.338 | 53998.5 |
| 2 | FERD3L | 49.195 | 26.187 | 19058.5 |
| 3 | U2AF2 | 23.008 | 3.434 | 8930 |
| 4 | SUV39H1 | 19.574 | 8.976 | 7602 |
| 5 | Hnmph1 | 10.598 | 5.005 | 4130 |
| 6 | HNRNPH1 | 5.593 | 0.518 | 2194.5 |
| 7 | DBN1 | 5.075 | 0.201 | 1994 |

CD1-M8

| 1 | IL1F10/IL38 | 142.421 | 91.494 | 45625.5 |
|---|---|---|---|---|
| 2 | FERD3L | 50.927 | 40.838 | 16334 |
| 3 | DBN1 | 10.089 | 4.376 | 3260 |
| 4 | ESYT2_frag | 5.713 | 2.152 | 1859 |
| 5 | CROCCP2 | 3.561 | 0.747 | 1170 |
| 6 | CROCCP2 | 2.814 | 1.068 | 931 |
| 7 | NDOR1 | 1.746 | 0.155 | 589 |
| Rank | Protein | Z-score | S-score | F635 |

NZB-M8

| | | | | |
|---|---|---|---|---|
| 1 | IFNG | 56.051 | 3.471 | 7518.5 |
| 2 | GORASP1 | 52.58 | 4.166 | 7056.5 |
| 3 | COL5A2 | 48.414 | 1.349 | 6502 |
| 4 | NEDD4L | 47.065 | 9.039 | 6322.5 |
| 5 | TTC5 | 38.026 | 3.689 | 5119.5 |
| 6 | LBHD1 | 34.337 | 0.297 | 4628.5 |
| 7 | IL1F10/IL38 | 34.04 | 0.383 | 4589 |
| 8 | FERD3L | 33.657 | 1.056 | 4538 |

NZB-M8, however, identified IFNγ as the top hit and IL-38 as the 7th hit, suggesting low fidelity to IL-38. Since IL-38 is an IL-1 family member, antibody binding was also compared with other IL-1 family members. Despite homology between IL-38 and other IL-1 family members, no cross-reactivity was identified (Table 5).

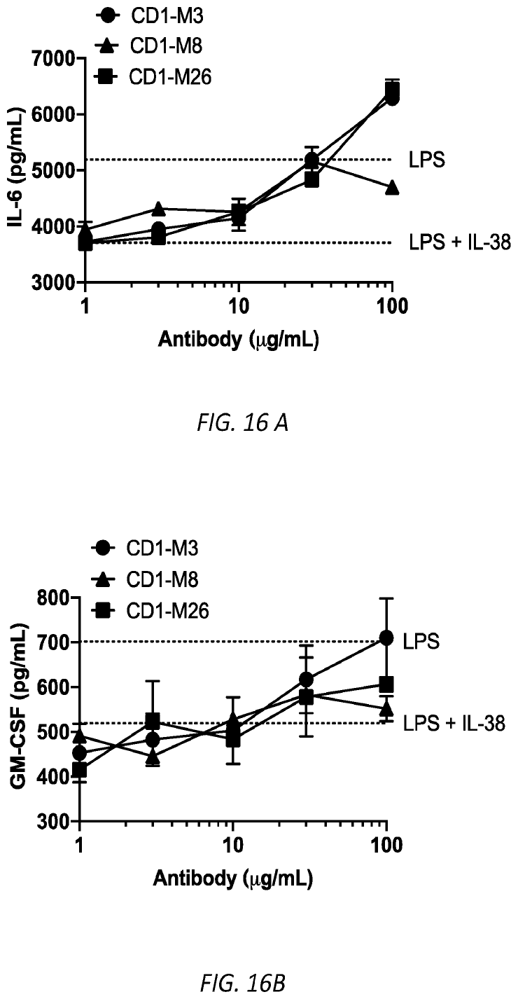

CD1-M3, CD1-M8, and CD1-M26 were isolated from monoclonal antibody supernatants and PBS-purified. These antibodies were tested in the established in vitro system described in FIG. 11 using half log dilutions of each antibody. IL-6 and GM-CSF production was assessed using Human IL-6 and Human GM-CSF DuoSet ELISA Kits (R&D Systems) per the manufacturer's instructions. All lead antibodies were capable of restoring IL-6 and GM-CSF production of IL-38-treated, LPS-stimulated THP-1 macrophages (FIGS. 16A-B).

TABLE 5
Binding of lead candidate antibodies to IL-1
family members in protein microarray format.

| IL-1 Family | CD1-M3 | CD1-M8 | NZB-M8 |
|---|---|---|---|
| Member | (Z-Score) | (Z-score) | (Z-score) |
| IL-38 | 139.533 | 142.421 | 34.04 |
| IL-1α | −0.013 | −0.014 | −0.147 |
| IL-1β | −0.016 | −0.017 | −0.143 |
| IL-1Ra | −0.013 | 0.003 | −0.166 |
| IL-18 | −0.016 | −0.016 | −0.132 |
| IL-36Ra | −0.017 | −0.019 | −0.034 |
| IL-36a | −0.008 | 0.012 | 0.033 |
| IL-37 | −0.013 | −0.016 | −0.113 |
| IL-36b | −0.016 | −0.01 | −0.087 |
| IL-36g | −0.017 | −0.008 | −0.143 |
| IL-33 | −0.02 | −0.014 | −0.17 |

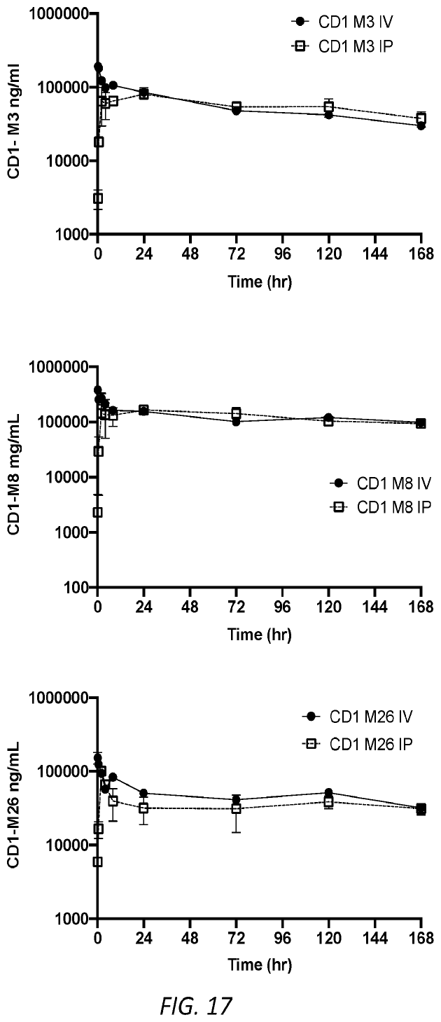

Example 6. Evaluating the effect of anti-IL-38 antibodies in in vivo tumor models. After CD1-M3, CD1-M8, and CD1-M26 were confirmed to bind and block IL-38 function in vitro, they were assessed for their ability to persist in mouse plasma in a pharmacokinetic study. 6-7 week old C57BL/6 mice were dosed at 10 mg/kg i.p. and i.v. at 0 h (n=9 per group). Each mouse was retro-orbitally bled for two timepoints and terminally bled at a final timepoint. Plasma was isolated using K2 EDTA tubes and assayed for antibody via direct IL-38 ELISA. 100 μL of IL-38 in PBS (50 ng/mL for CD1-M3, M8; 600 ng/mL for CD1-M26) was added per well of a 96 well high binding plate and plates were incubated overnight at 4° C. After 3 washes with PBS 0.05% Tween, plates were blocked with PBS 2% BSA for 1 h at room temperature. After 3 washes, 100 μL mouse plasma diluted in PBS 2% BSA was added each well. To generate a standard curve, CD1-M3, M8, M26 antibodies were spiked into untreated mouse plasma diluted with PBS 2% BSA starting at 500 ng/mL. Plates were incubated for 2 h at room temperature and washed 3 times. 100 μL of HRP-conjugated anti-mouse antibody diluted 1:2000 in PBS 2% BSA was added per well and incubated for 2 h at room temperature. After 3 washes, 100 μL of OPD substrate diluted in phospho-citrate/sodium perborate buffer was added per well, developed for 5-30 minutes, and absorbance was measure at 450 nm. After a 10 mg/kg dose, all antibodies reached 100,000 ng/mL plasma concentration soon after dosing, which slowly decreased over time (FIG. 17). i.v. and i.p. administration of CD1-M3, CD1-M8, and CD1-M26 resulted in similar plasma levels throughout the one week study.

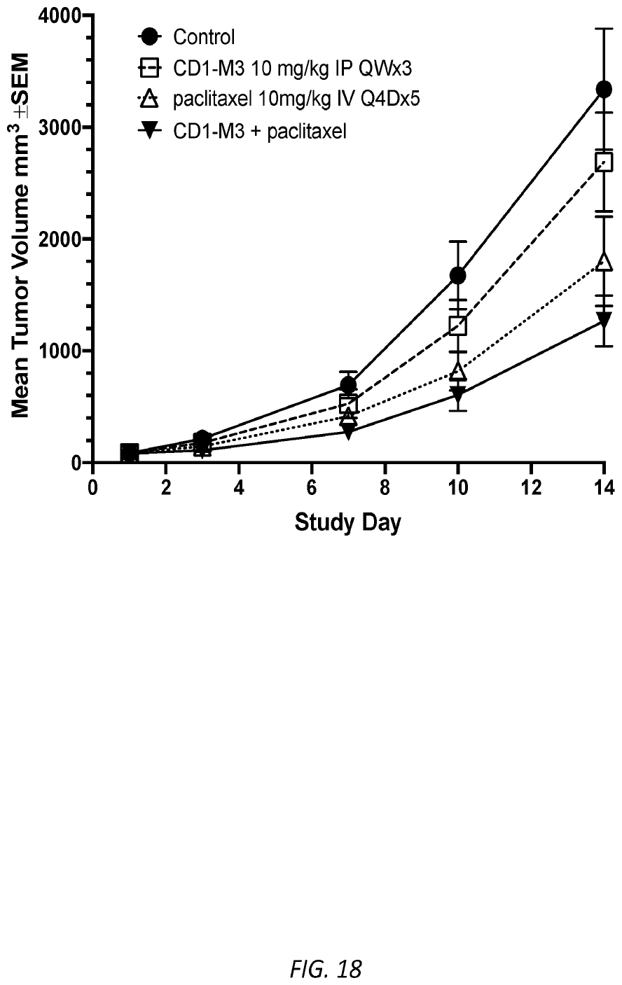

Of the selected lead candidate antibodies, CD1-M3 was the only antibody capable of binding human and mouse IL-38. Therefore, this antibody was tested in several syngeneic tumor models, where blocking IL-38 in the tumor microenvironment could be evaluated in an immune competent mouse. In the first study, 2×10⁵ B16.F10 cells were implanted into the flank of 6-8 week old C57BL/6 female mice. When mean tumor size reached 85 mm³, mice were randomized into indicated groups (n=10). Mice were treated with CD1-M3 and/or paclitaxel as indicated and tumor volumes were taken 3 times per week with a caliper. Treatment with anti-CD1-M3 resulted in a small reduction in tumor volume compared with vehicle control (FIG. 18). CD1-M3 treatment also reduced tumor volume when combined with the chemotherapeutic agent paclitaxel.

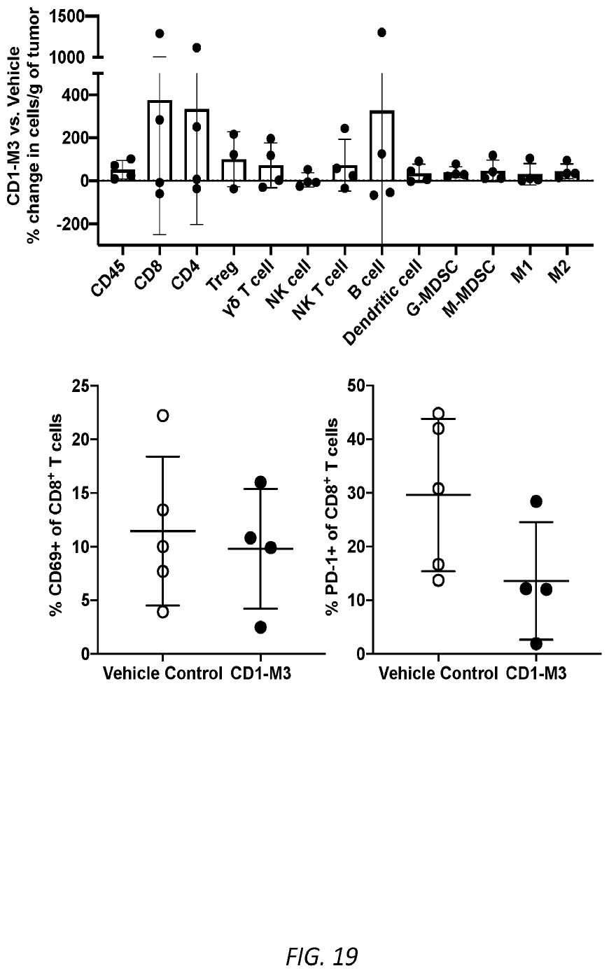

Due to the reported role of IL-38 in suppressing inflammatory immune responses, an additional study was performed to evaluate the effect that CD1-M3 would have on the tumor infiltrating myeloid and lymphocyte populations. 2×10⁵ B16.F10 cells were implanted into the flank for 7-8 week old C57BL/6 female mice. When mean tumor size reached 104 mm³, mice were randomized into Vehicle and CD1-M3-treated groups and dosed at 10 mg/mL IP QWx2. On Day 9, 24 hours after the 2^{nd} dose, mice were euthanized and tumors were dissociated into single cell suspensions. Lymphocyte and myeloid populations were assessed using two flow cytometry panels. T cell panel includes Zombie NIR Viability dye (Biolegend) and fluorochrome-conjugated antibodies recognizing CD3, CD4, CD8, CD45, CD25, PD-1, CD69, FoxP3, CD49b/CD335, TCRgd. Myeloid panel includes Zombie NIR Viability dye (Biolegend) and fluorochrome-conjugated antibodies recognizing CD45, CD11b, CD11c, CD24, Ly-6C, Ly-6G, F4/80, MHCII, and CD206. Cell number was calculated using Precision Count beads (BioLegend) and normalized based on tumor size. All populations were gated on single, live CD45+ cells. Populations are defined as follows: Treg—CD4*CD25*FoxP3+; NK cell—CD3⁻CD49b+CD335+; NKT cell—CD3+CD49b+CD335+; G-MDSC—CD11b+Ly6G+; M-MDSC—CD11b+Ly6C+; Macrophage—CD11b+F4/80+(exclude MDSC); M1—CD206-MHCII+ Macrophages; M2—CD206+ Macrophages; Dendritic cells—CD24+F4/80-CD11c+MHCII+. Percent change was calculated as [(cells/gram of CD1-M3 sample)−(cells/gram of Vehicle control group)]/(cells/gram of Vehicle control group)×100%. Notably, CD1-M3 treatment resulted in an increase in the tumor infiltrating T cells, including CD4, CD8, and γδ T cells (FIG. 19, top). The B cell population was also increased compared with vehicle control. CD1-M3 did not affect the expression of the activation marker CD69 on intratumoral CD8 T cells, however, it did decrease the amount of CD8 T cells expressing PD-1 (FIG. 19, bottom).

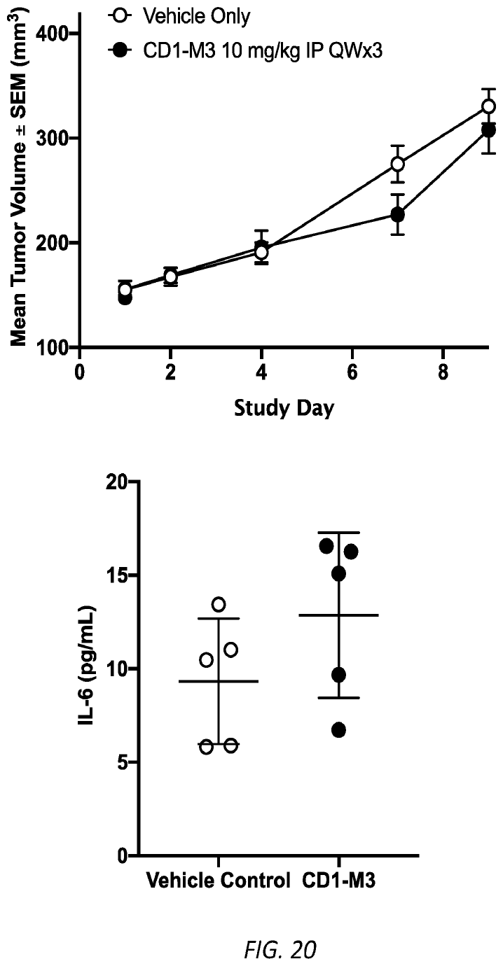

The second syngeneic model evaluated was the MMTV-PyMT orthotopic mouse model. 10⁶ MMTV-PyMT cells were implanted orthotopically into the mammary fat pad of female FVB mice. When mean tumor size reached 150 mm³, mice were randomized into groups (n=10) and dosed as indicated. Tumor volumes were measured with a caliper every 2-3 days and study was concluded on Day 9, 24 hours after the 2^{nd} dose. CD1-M3 treatment again resulted in a small reduction in tumor volume compared with the vehicle control (FIG. 20, top). Since CD1-M3 can restore IL-6 production of IL-38-treated macrophages in vitro, intratumor cytokine levels were evaluated in this study. Flash frozen tumors were homogenized in lysis buffer containing 0.5% NP-40 using a bead beating homogenizer (Omni International). Protein concentration was measured by Pierce BCA Protein Assay Kit (ThermoFisher) and normalized. Cytokine concentration was measured on a Luminex-based platform. Similar to in vitro data showing restoration of IL-6 production with CD1-M3, intratumoral IL-6 was increased in CD1-M3-treated mice.

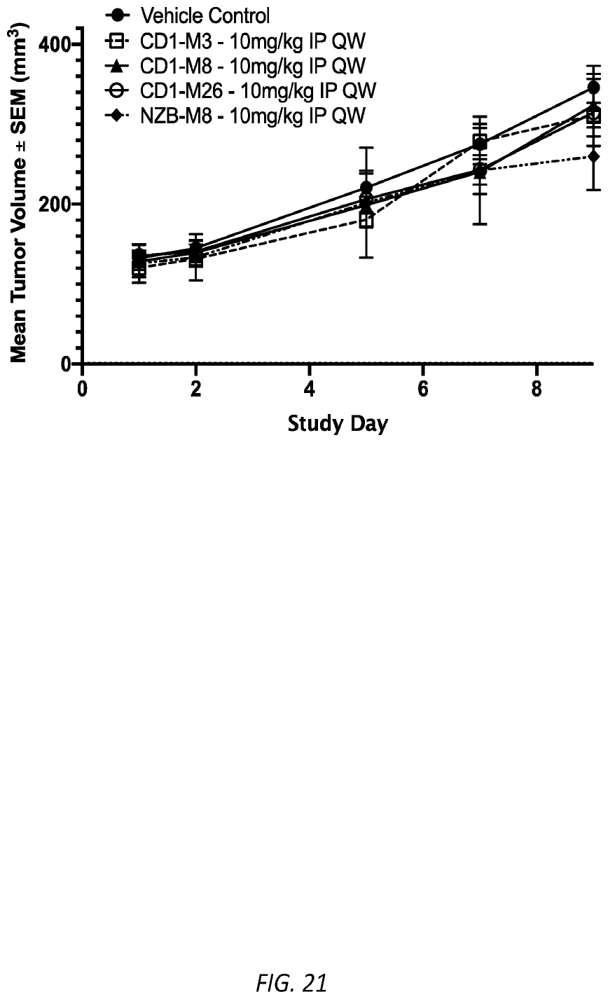

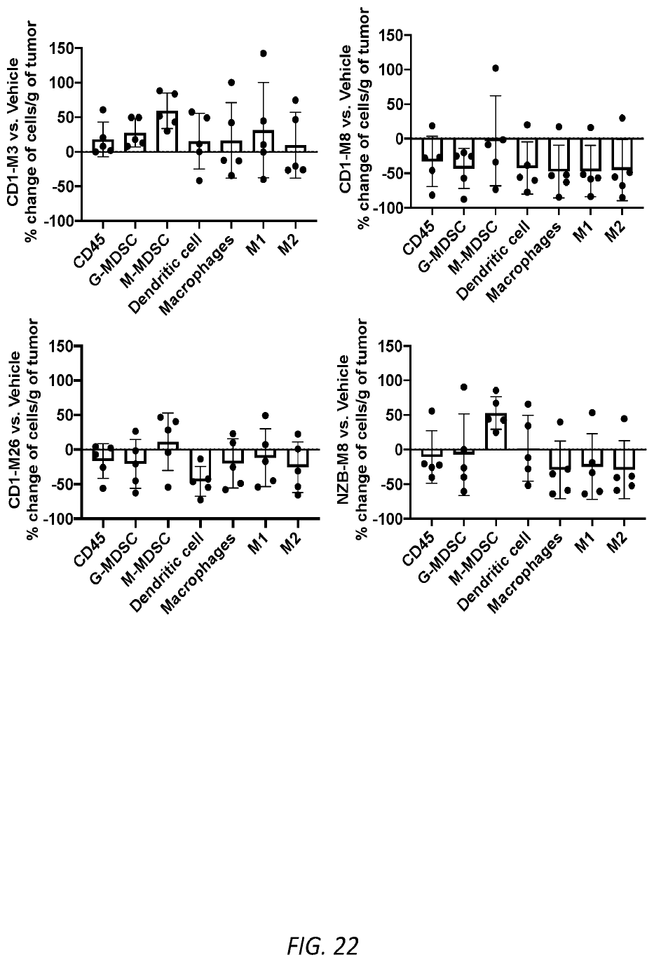

To evaluate additional lead candidate antibodies that only bind human IL-38, a xenograft model was also evaluated in immune-deficient scid mice. 5×10⁶ A549 cells, which have been previously shown to secrete IL-38 under apoptotic conditions, were implanted into 7-8 week old female scid mice. When mean tumor size reached 134 mm³, mice were randomized into groups and dosed as indicated. Tumor volumes were measured with a caliper every 2-3 days and study was concluded on Day 9, 24 hours after the 2^{nd} dose. No major reduction in tumor volume was observed after treatment the lead candidate antibodies (FIG. 21). This may be due to the lack of T and B cells in this model, which were increased in B16.F10 tumors (FIG. 19). The myeloid compartment, which is still present in scid mice, was evaluated in these mice by harvesting the tumors from FIG. 21 and dissociating them into single cell suspensions and flow cytometry was performed as described in FIG. 19. Overall, CD1-M3 slightly increased multiple myeloid populations, while CD1-M8, CD1-M26, and NZB-M8 largely resulted in a slight decrease in these populations (FIG. 22).

What is claimed:

1. An isolated Interleukin-38 (IL-38)-binding antibody, or antigen-binding fragment thereof, comprising at least 3, at least 4, at least 5, at least 6, at least 7, or at least 8 optionally contiguous amino acids of at least one complementary-determining region (CDR) contained within the variable heavy chain (VH) amino acid sequences of: SEQ ID NO: 22, SEQ ID NO: 27, SEQ ID NO: 32, SEQ ID NO: 37, SEQ ID NO: 42, SEQ ID NO: 47, SEQ ID NO: 52, SEQ ID NO: 2, or SEQ ID NO: 7; and/or contained within the variable light chain (VH) amino acid sequences of: SEQ ID NO: 57, SEQ ID NO: 62, SEQ ID NO: 67, SEQ ID NO: 72, SEQ ID NO: 77, SEQ ID NO: 82, SEQ ID NO: 4, or SEQ ID NO: 9

2. The isolated antibody, or antigen-binding fragment thereof, wherein the CDRs are defined by the North method or by the Kabat method.

3. An antibody, or antigen-binding fragment thereof, comprising:
at least 3, at least 4, at least 5, at least 6, at least 7, or at least 8 amino acids of:
the VH CDR1 are selected from the amino acid sequences of SEQ ID NOS: 23, 28, 33, 38, 43, 48, 53, or 15;
the VH CDR2 are selected from the amino acid sequences of SEQ ID NOS: 24, 29, 34, 39, 44, 49, 54, or 16;
the VH CDR3 are selected from the amino acid sequences of SEQ ID NOS: 25, 30, 35, 40, 45, 50, 55, or 17;
the VL CDR1 are selected from the amino acid sequences of SEQ ID NOS: 58, 63, 68, 73, 78, 83, or 18;
the VL CDR2 are selected from the amino acid sequences of SEQ ID NOS: 59, 64, 69, 74, 79, 84, or 19; and
the VL CDR3 are selected from the amino acid sequences of SEQ ID NOS: 60, 65, 70, 75, 80, 85, or 20.

4. The antibody, or antigen-binding fragment thereof, of claim 3, comprising at least one CDR selected from: a VH CDR1 of SEQ ID NO: 33, a VH CDR2 of SEQ ID NO: 34, a VH CDR3 of SEQ ID NO: 35, a VL CDR1 of SEQ ID NO: 63, a VL CDR2 of SEQ ID NO: 64, and a VL CDR3 of SEQ ID NO: 65.

5. The antibody, or antigen-binding fragment thereof, of claim 3, comprising at least one CDR selected from: a VH CDR1 of SEQ ID NO: 38, a VH CDR2 of SEQ ID NO: 39, a VH CDR3 of SEQ ID NO: 40, a VL CDR1 of SEQ ID NO: 68, a VL CDR2 of SEQ ID NO: 69, and a VL CDR3 of SEQ ID NO: 70.

6. The antibody, or antigen-binding fragment thereof, of claim 3, comprising at least one CDR selected from: a VH CDR1 of SEQ ID NO: 43, a VH CDR2 of SEQ ID NO: 44, a VH CDR3 of SEQ ID NO: 45, a VL CDR1 of SEQ ID NO: 73, a VL CDR2 of SEQ ID NO: 74, and a VL CDR3 of SEQ ID NO: 75.

7. The antibody, or antigen-binding fragment thereof, of claim 3, comprising at least one CDR selected from: a VH CDR1 of SEQ ID NO: 48, a VH CDR2 of SEQ ID NO: 49, a VH CDR3 of SEQ ID NO: 50, a VL CDR1 of SEQ ID NO: 78, a VL CDR2 of SEQ ID NO: 79, and a VL CDR3 of SEQ ID NO: 80.

8. The antibody, or antigen-binding fragment thereof, of claim 3, comprising at least one CDR selected from: a VH CDR1 of SEQ ID NO: 53, a VH CDR2 of SEQ ID NO: 54, a VH CDR3 of SEQ ID NO: 55, a VL CDR1 of SEQ ID NO: 83, a VL CDR2 of SEQ ID NO: 84, and a VL CDR3 of SEQ ID NO: 85.

9. The antibody, or antigen-binding fragment thereof, of claim 3, comprising at least one CDR selected from: SEQ ID NO: 15, a VH CDR2 of SEQ ID NO: 16, a VH CDR3 of SEQ ID NO: 17, a VL CDR1 of SEQ ID NO: 18, a VL CDR2 of SEQ ID NO: 19, and a VL CDR3 of SEQ ID NO: 20

10. The antibody, or antigen-binding fragment of any one of claims 1-9, wherein the IL-38 is a component of a multi-protein complex.

11. The antibody, or antigen-binding fragment of any one of claims 1-10, wherein the antibody, or antigen-binding fragment partially or fully blocks, inhibits, or neutralizes a biological activity of IL-38.

12. The antibody or antigen-binding fragment of any one of claims 1-11, wherein the IL-38 is present in a body fluid.

13. The antibody or antigen-binding fragment of claim 12, wherein the body fluid is blood or a blood derivative.

14. The antibody or antigen-binding fragment of claim 13, wherein the blood derivative is plasma or serum.

15. The antibody or antigen-binding fragment of any one of claims 1-14, wherein the IL-38 is associated with an extracellular matrix (“ECM”), or ECM protein.

16. The antibody or antigen-binding fragment of claim 15, wherein the IL-38 is present in a tumor micro-environment.

17. The antigen-binding fragment of any one of claims 1-16, wherein the antigen-binding fragment is an isolated variable heavy (VH) single domain monoclonal antibody.

18. The antigen-binding fragment of any one of claims 1-16, wherein the antigen-binding fragment is a single chain (sc)Fv-Fc fragment.

19. The antigen-binding fragment of any one of claims 1-16, wherein the isolated antigen-binding fragment comprises an Fv, scFv, Fab, F(ab′)2, or Fab′ fragment, diabody, or any fragment whose half-life may have been increased.

20. The antibody or antigen-binding fragment of any one of the claims 1-19, wherein the antibody or antigen-binding fragment comprises a CH3 scaffold, comprising at least one modification of the wild-type amino acid sequence of the CH3 domain derived from an immunoglobulin Fc region.

21. The antibody or antigen-binding fragment of any one of claims 1-20, wherein the antibody or antigen-binding fragment is monoclonal.

22. The antibody or antigen-binding fragment of any one of claims 1-21, wherein the antibody or antigen-binding fragment is human, humanized, or bi-specific.

23. A method of inhibiting tumor growth or metastasis in a subject, comprising administering to the subject a therapeutically effective amount of a composition comprising the antibody or an antigen-binding fragment of any of claims 1-22, wherein the antibody or an antigen-binding fragment partially or fully blocks, inhibits, or neutralizes a biological activity of IL-38.
